(19) United States
(12) Patent Application Publication
(10) Pub. No.: US 2025/0388663 A1
(43) Pub. Date: Dec. 25, 2025

(54) IMPROVED ANTI-ALBUMIN NANOBODIES AND THEIR USES

(71) Applicants: INSTITUT NATIONAL DE LA SANTÉ ET DE LA RECHERCHE MÉDICALE, Paris (FR); UNIVERSITÉ PARIS-SACLAY, Gif-sur-Yvette (FR)

(72) Inventors: Petrus LENTING, Le Kremlin-Bicêtre (FR); Olivier CHRISTOPHE; Cécile DENIS, Le Kremlin-Bicêtre (FR); Ivan PEYRON, Le Kremlin-Bicêtre (FR); Caterina CASARI, Le Kremlin-Bicêtre (FR)

(21) Appl. No.: 19/103,875

(22) PCT Filed: Aug. 16, 2023
(86) PCT No.: PCT/EP2023/072615

§ 371 Date: Feb. 14, 2025

(30) Foreign Application Priority Data
Nov. 15, 2022 (EP) 22306681.2
Aug. 17, 2023 (EP) 22306232.4

(51) Int. Cl. C07K 16/18; A61K 39/00; A61P 7/04; C07K 16/36

(52) U.S. Cl. CPC C07K 16/18; A61P 7/04; C07K 16/36; A61K 2039/505; C07K 2317/24; C07K 2317/31; C07K 2317/33; C07K 2317/569; C07K 2317/92; C07K 2317/94

(57) ABSTRACT

Via yeast display technology, albumin binding variants were selected, and variants displayed improved binding to MSA or both HSA & MSA. All variants were subcloned into the pHEN-expression plasmid and expressed in E coli WK6 cells. Purified nanobodies were then analyzed for their capacity to bind immobilized MSA. Biotinylated nanobodies were then tested in vivo for their circulatory half-life. They then validated the effect of their nanobodies anti albumin linked with another nanobodies such as single-domain antibody anti-D′D3 as described in WO2017129630 and WO2018091621.

Mice having reduced levels of VWF (Von Willebrand disease type-1) received a single dose of the bispecific single-domain antibody. VWF and FVIII levels were followed over 14 days. These data show that there is a statistically significant increase in VWF and FVIII levels, which is maintained for 10 days.

The present invention relates to an isolated single-domain antibody (sdAb) directed against albumin and its uses in different diseases such as bleeding disorders.

### FIELD OF THE INVENTION

The invention relates to anti-albumin nanobodies and their uses for therapy.

### BACKGROUND OF THE INVENTION

In certain diseases, a natural or endogenous protein is defective or missing in the patient, in particular because of inherited gene defects. In other diseases, the level of the natural or endogenous protein is not enough to have a normal function in the patient compared to a healthy person, the low of said endogenous protein is lower in the patient than in a healthy subject.

There are many methods to overcoming these problems. Particularly, the use of polypeptides such as proteins for therapeutic applications has expanded in recent years mainly due to advanced knowledge of the molecular biological principles underlying many diseases and the availability of improved recombinant expression and delivery systems for human polypeptides. In the prior art, the short circulating half-life of polypeptide therapeutics has been addressed by covalent attachment of a polymer to the polypeptide. However, a number of problems have been observed with the attachment of polymers. For example, the attachment of polymers can lead to decreased drug activity. Furthermore, certain reagents used for coupling polymers to a protein are insufficiently reactive and therefore require long reaction times during which protein denaturation and/or inactivation can occur. Also, incomplete or non-uniform attachment leads to a mixed population of compounds having differing properties.

However, there are few methods in the art to increase the half-life and the level of endogenous or exogenous proteins which are defective, missing or not enough to function correctly (WO2018091621).

Single domain antibodies (sdAbs) or nanobodies or VHH targeting albumin are well-known in the art. By binding to both human and murine albumin, anti-albumin nanobodies as described in the art (such as ALB8; SEQ ID NO: 1, described in WO2006/122787) can be used in the pre-clinical mouse models before moving on to primates and human studies. However, binding of ALB8 to murine albumin is relatively weak (half-maximal binding of ALB8 to MSA requires 35-fold higher sdAb concentrations compared to binding to HSA).

Thus, there is still a need for anti-albumin nanobodies that increase the half-life and the level of the endogenous or exogenous proteins to increase efficiency or reduce the amount of therapeutic proteins and/or frequency of infusions applied to patient. This would also reduce the costs of the treatment.

### SUMMARY OF THE INVENTION

The invention relates to an isolated single-domain antibody (sdAb) directed against albumin comprising:
a CDR1 having a sequence set forth as SEQ ID NO: 2, a CDR2 having a sequence set forth as SEQ ID NO: 3 and a CDR3 having a sequence set forth as SEQ ID NO: 4;
a CDR1 having a sequence set forth as SEQ ID NO:6, a CDR2 having a sequence set forth as SEQ ID NO: 7 and a CDR3 having a sequence set forth as SEQ ID NO: 8;
a CDR1 having a sequence set forth as SEQ ID NO:10, a CDR2 having a sequence set forth as SEQ ID NO: 11 and a CDR3 having a sequence set forth as SEQ ID NO: 12;
a CDR1 having a sequence set forth as SEQ ID NO:14, a CDR2 having a sequence set forth as SEQ ID NO: 15 and a CDR3 having a sequence set forth as SEQ ID NO: 16; or
a CDR1 having a sequence set forth as SEQ ID NO:18, a CDR2 having a sequence set forth as SEQ ID NO: 19 and a CDR3 having a sequence set forth as SEQ ID NO: 20.

In particular, the invention is defined by claims.

### DETAILED DESCRIPTION OF THE INVENTION

Single domain antibodies (sdAbs) or nanobodies targeting albumin are well-known in the art. By binding to both human and murine albumin, anti-albumin nanobodies as described in the art (such as ALB8; SEQ ID NO: 1, described in WO2006/122787) can be used in the pre-clinical mouse models before moving on to primates and human studies. However, binding of ALB8 to murine albumin is relatively weak (half-maximal binding of ALB8 to MSA requires 35-fold higher sdAb concentrations compared to binding to HSA).

SEQ ID NO: 1
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSS.

Inventors have therefore decided to go through an affinity maturation step. They generated two libraries. In the first library, each amino acid in the CDR1, CDR2 and CDR3 was replaced by one of 19 possible other amino acids (with the exception of cysteine). In the second library, the combined CDR sequences contained one, two or three mutations, with 19 possible amino acid replacements for each amino acid in the three CDRs.

Sequences were amplified by PCR and recombined into the yeast display plasmid pSTALK-Halo to create a library with a complexity of 703 yeast cells for the simple variant library and 3.62 million yeast cells for the simple, double and triple variant library. 48 random clones of each libraries have been sequenced to validate the mutagenesis conditions. To facilitate cloning into the yeast-expression system, the N-terminal portion of the framework sequence was modified from EVOLVESGGGLV (SEQ ID NO: 41) into QVQLQQSGGGFV (SEQ ID NO: 42) (changed amino acids in bold). This modified sequence was selectively used in the yeast-display system.

Via yeast display technology, albumin binding variants were selected, and variants displayed improved binding to MSA. All variants were subcloned into the pHEN-expression plasmid (having now the original EVOLVESGGGLV N-terminal sequence) and expressed in E coli WK6 cells. Purified nanobodies were then analysed for their capacity to bind immobilized MSA. Biotinylated nanobodies were then tested in vivo for their circulatory half-life. They then validated the effect of their nanobodies anti albumin linked with other nanobodies such as single-domain antibody anti-D′D3 as described in WO2017129630 and WO2018091621.

Mice having reduced levels of VWF (Von Willebrand disease type-1) received a single dose of the bispecific single-domain antibody. VWF and FVIII levels were followed over 14 days. These data show that there is a statistically significant increase in VWF and FVIII levels, which is maintained for 10 days.

### Single-Domain Antibodies Directed Against Albumin

Accordingly, in a first aspect, the invention relates to an isolated single-domain antibody (sdAb) directed against albumin comprises:
a CDR1 having a sequence set forth as SEQ ID NO: 2, a CDR2 having a sequence set forth as SEQ ID NO: 3 and a CDR3 having a sequence set forth as SEQ ID NO: 4;
a CDR1 having a sequence set forth as SEQ ID NO:6, a CDR2 having a sequence set forth as SEQ ID NO: 7 and a CDR3 having a sequence set forth as SEQ ID NO: 8;
a CDR1 having a sequence set forth as SEQ ID NO:10, a CDR2 having a sequence set forth as SEQ ID NO: 11 and a CDR3 having a sequence set forth as SEQ ID NO: 12;
a CDR1 having a sequence set forth as SEQ ID NO:14, a CDR2 having a sequence set forth as SEQ ID NO: 15 and a CDR3 having a sequence set forth as SEQ ID NO: 16; or
a CDR1 having a sequence set forth as SEQ ID NO:18, a CDR2 having a sequence set forth as SEQ ID NO: 18 and a CDR3 having a sequence set forth as SEQ ID NO: 20.

In another embodiment, the isolated single-domain antibody directed against albumin according to the invention, wherein said single-domain antibody comprises:
a CDR1 having at least 70% of identity with sequence set forth as SEQ ID NO: 2, a CDR2 having at least 70% of identity with sequence set forth as SEQ ID NO: 3 and a CDR3 having at least 70% of identity with sequence set forth as SEQ ID NO: 4;
a CDR1 having at least 70% of identity with sequence set forth as SEQ ID NO: 6, a CDR2 having at least 70% of identity with sequence set forth as SEQ ID NO: 7 and a CDR3 having at least 70% of identity with sequence set forth as SEQ ID NO: 8;
a CDR1 having at least 70% of identity with sequence set forth as SEQ ID NO: 10, a CDR2 having at least 70% of identity with sequence set forth as SEQ ID NO: 11 and a CDR3 having at least 70% of identity with sequence set forth as SEQ ID NO: 12;
a CDR1 having at least 70% of identity with sequence set forth as SEQ ID NO: 14, a CDR2 having at least 70% of identity with sequence set forth as SEQ ID NO: 15 and a CDR3 having at least 70% of identity with sequence set forth as SEQ ID NO: 16; or
a CDR1 having at least 70% of identity with sequence set forth as SEQ ID NO: 18, a CDR2 having at least 70% of identity with sequence set forth as SEQ ID NO: 19 and a CDR3 having at least 70% of identity with sequence set forth as SEQ ID NO: 20.

In a further embodiment, the isolated single-domain antibody directed against albumin according to the invention wherein said single-domain antibody is:
OptiAlb-03 (SEQ ID NO: 5),
OptiAlb-07 (SEQ ID NO:9);
OptiAlb-09 (SEQ ID NO:13);
OptiAlb-11 (SEQ ID NO:17) or
OptiAlb-12 (SEQ ID NO:21).

As used herein the term “single-domain antibody” (sdAb) has its general meaning in the art and refers to the single heavy chain variable domain of antibodies of the type that can be found in Camelid mammals which are naturally devoid of light chains. Such single-domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al, Trends Biotechnol, 2003, 21(11): 484-490; and WO 06/030220, WO 06/003388. The amino acid sequence and structure of a single-domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementary Determining Region 1” or “CDR1”; as “Complementarity Determining Region 2” or “CDR2” and as “Complementarity Determining Region 3” or “CDR3”, respectively. Accordingly, the single-domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single-domain antibody are numbered according to the general numbering for VH domains given by the International ImMunoGeneTics information system amino acid numbering (http:/imgt.cines.fr/).

As used herein, the term “amino acid sequence” has its general meaning and is a sequence of amino acids that confers to a protein its primary structure. According to the invention, the amino acid sequence may be modified with one, two or three conservative amino acid substitutions, without appreciable loss of interactive binding capacity. By “conservative amino acid substitution”, it is meant that an amino acid can be replaced with another amino acid having a similar side chain. Families of amino acid having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., glycine, cysteine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with the second amino acid sequence. Amino acid sequence identity is typically determined using a suitable sequence alignment algorithm and default parameters, such as BLAST P (Karlin and Altschul, 1990).

According to the meaning of the present invention, the “identity” is calculated by comparing two aligned sequences in a comparison window. The sequence alignment allows determining the number of positions (nucleotides or amino acids) in common for the two sequences in the comparison window. The number of positions in common is therefore divided by the total number of positions in the comparison window and multiplied by 100 to obtain the identity percentage. The determination of the identity percentage of sequence can be made manually or thanks to well-known computer programs.

As used herein, the terms “purified” and “isolated” relate to the sdAb of the invention and mean that the sdAb is present in the substantial absence of other biologic macromolecules of the same type. The term “purified” as used here means preferably that at least 75% in weight, more preferably at least 85% in weight, even more preferably at least 95% in weight, and the more preferably at least 98% in weight of antibody, compared to the total weight of macromolecules present.

As used herein, the term “nucleic acid molecule” has its general meaning in the art and refers to a DNA or RNA molecule.

As used herein, the term “albumin” refers to a transport protein that bind to various ligands and carry them around. Albumin is found in blood plasma and differs from other blood proteins in that it is not glycosylated. In a particular embodiment, the albumin is human serum albumin (HSA) which is found in human blood. The naturally occurring human HSA gene has a nucleotide sequence as shown in Genbank Accession number NM_000477 and the naturally occurring human HSA protein has an aminoacid sequence as shown in Genbank Accession number NP_000468, SEQ ID NO:71. The murine nucleotide and amino acid sequences have also been described (Genbank Accession numbers NM_009654 and NP_033784).

Human HSA protein
SEQ ID NO: 71
MKWVTFISLLFLFSSAYSRGVFRRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDRADLAKYICENQDSISSKLK
ECCEKPLLEKSHCIAEVENDEMPADLPSLAADFVESKDVCKNYAEAKDVF
LGMFLYEYARRHPDYSVVLLLRLAKTYETTLEKCCAAADPHECYAKVFDE
FKPLVEEPQNLIKQNCELFEQLGEYKFQNALLVRYTKKVPQVSTPTLVEV
SRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASQAALGL.

Inventors have isolated five single-domain antibodies (sdAb) with the required properties and characterized the complementarity determining regions (CDRs) of said sdAb and thus determined the CDRs of said sdAb (Table A):

TABLE A
Sequences of sdAb anti-albumin domains.

| OptiAlb-03 | Sequences |
|---|---|
| CDR1 | SEQ ID NO: 2 |
| | GFTFSSFG |
| CDR2 | SEQ ID NO: 3 |
| | ISGSGSDT |
| CDR3 | SEQ ID NO: 4 |
| | TIGGSLSPSSQ |
| SEQUENCE | SEQ ID NO: 5 |
| OptiAlb-03 | EVQLVESGGGLVQPGNSLRLSCAASGFTFSSFGMSWVRQAPGK |
| | GLEWVSSISGSGSDTLYADSVKGRFTISRDNAKTTLYLQMNSLRP |
| | EDTAVYYCTIGGSLSPSSQGTLVTVSS |
| OptiAlb-07 | Sequences |
| CDR1 | SEQ ID NO: 6 |
| | GFTFSSFG |
| CDR2 | SEQ ID NO: 7 |
| | ISGSGDDT |
| CDR3 | SEQ ID NO: 8 |
| | TIGGSLSPSSQ |
| SEQUENCE | SEQ ID NO: 9 |
| OptiAlb-07 | EVQLVESGGGLVQPGNSLRLSCAASGFTFSSFGMSWVRQAPGK |
| | GLEWVSSISGSGDDTLYADSVKGRFTISRDNAKTTLYLQMNSLR |
| | PEDTAVYYCTIGGSLSPSSQGTLVTVSS |
| | (Bold: CDR sequence and not bold: framework sequence) |
| OptiAlb-09 | Sequences |
| CDR1 | SEQ ID NO: 10 |
| | GFTFSSFG |
| CDR2 | SEQ ID NO: 11 |
| | ISGSGSDT |
| CDR3 | SEQ ID NO: 12 |
| | TIGGSKSRSSG |
| SEQUENCE | SEQ ID NO: 13 |
| OptiAlb-09 | EVQLVESGGGLVQPGNSLRLSCAASGFTFSSFGMSWVRQAPGK |
| | GLEWVSSISGSGSDTLYADSVKGRFTISRDNAKTTLYLQMNSLRP |
| | EDTAVYYCTIGGSKSRSSGGTLVTVSS |
| | (Bold: CDR sequence and not bold: framework sequence) |
| OptiAlb-11 | Sequences |
| CDR1 | SEQ ID NO: 14 |
| | GFTFSSFG |
| CDR2 | SEQ ID NO: 15 |
| | ISGSGHDT |
| CDR3 | SEQ ID NO: 16 |
| | TIGGSLTRSSQ |
| SEQUENCE | SEQ ID NO: 17 |
| OptiAlb-11 | EVQLVESGGGLVQPGNSLRLSCAASGFTFSSFGMSWVRQAPGK |
| | GPEWVSSISGSGHDTLYADSVKGRFTISRDNAKTTLYLQMNSLR |
| | PEDTAVYYCTIGGSLTRSSQGTLVTVSS |
| | (Bold: CDR sequence and not bold: framework sequence) |
| OptiAlb-12 | Sequences |
| CDR1 | SEQ ID NO: 18 |
| | GFTFSRFG |
| CDR2 | SEQ ID NO: 19 |
| | ISGSGSDT |
| CDR3 | SEQ ID NO: 20 |
| | TIGGSLSPSSQ |
| SEQUENCE | SEQ ID NO: 21 |
| OptiAlb-12 | EVQLVESGGGLVQPGNSLRLSCAASGFTFSRFGMSWVRQAPGK |
| | GLEWVSSISGSGSDTLYADSVKGRFTISRDNAKTTLYLQMNSLRP |
| | EDTAVYYCTIGGSLSPSSQGTLVTVSS |
| | (Bold: CDR sequence and not bold: framework sequence) |

In a particular embodiment, the invention relates to an isolated single-domain antibody (sdAb OptiAlb-03) comprising a CDR1 having at least 80%, preferably at least 90%, more preferably at least 95%, even more preferably at least 99% sequence identity with sequence set forth as SEQ ID NO: 2, a CDR2 having at least 80%, preferably at least 90%, more preferably at least 95%, even more preferably at least 99% sequence identity with sequence set forth as SEQ ID NO: 3 and a CDR3 having at least 80%, preferably at least 90%, more preferably at least 95%, even more preferably at least 99% sequence identity with sequence set forth as SEQ ID NO: 4.

Amino acid sequence identity is preferably determined using a suitable sequence alignment algorithm and default parameters, such as BLAST P (Karlin and Altschul, Proc. Natl Acad. Sci. USA 87(6):2264-2268 (1990)).

In some embodiments, the isolated single-domain antibody according to the invention comprises a CDR1 having a sequence set forth as SEQ ID NO: 2, a CDR2 having a sequence set forth as SEQ ID NO: 3 and a CDR3 having a sequence set forth as SEQ ID NO: 4.

In some embodiments, the isolated single-domain antibody according to the invention has the sequence set forth as SEQ ID NO: 5.

In particular, the invention relates to an isolated single-domain antibody (sdAb OptiAlb-07) comprising a CDR1 having at least 80%, preferably at least 90%, more preferably at least 95%, even more preferably at least 99% sequence identity with sequence set forth as SEQ ID NO: 6, a CDR2 having at least 80%, preferably at least 90%, more preferably at least 95%, even more preferably at least 99% sequence identity with sequence set forth as SEQ ID NO: 7 and a CDR3 having at least 80%, preferably at least 90%, more preferably at least 95%, even more preferably at least 99% sequence identity with sequence set forth as SEQ ID NO: 8.

Amino acid sequence identity is preferably determined using a suitable sequence alignment algorithm and default parameters, such as BLAST P (Karlin and Altschul, Proc. Natl Acad. Sci. USA 87(6):2264-2268 (1990)).

In some embodiments, the isolated single-domain antibody according to the invention comprises a CDR1 having a sequence set forth as SEQ ID NO: 6, a CDR2 having a sequence set forth as SEQ ID NO: 7 and a CDR3 having a sequence set forth as SEQ ID NO: 8.

In some embodiments, the isolated single-domain antibody according to the invention has the sequence set forth as SEQ ID NO: 9.

In particular, the invention relates to an isolated single-domain antibody (sdAb OptiAlb-09) comprising a CDR1 having at least 80%, preferably at least 90%, more preferably at least 95%, even more preferably at least 99% sequence identity with sequence set forth as SEQ ID NO: 10, a CDR2 having at least 80%, preferably at least 90%, more preferably at least 95%, even more preferably at least 99% sequence identity with sequence set forth as SEQ ID NO: 11 and a CDR3 having at least 80%, preferably at least 90%, more preferably at least 95%, even more preferably at least 99% sequence identity with sequence set forth as SEQ ID NO: 12.

Amino acid sequence identity is preferably determined using a suitable sequence alignment algorithm and default parameters, such as BLAST P (Karlin and Altschul, Proc. Natl Acad. Sci. USA 87 (6): 2264-2268 (1990)).

In some embodiments, the isolated single-domain antibody according to the invention comprises a CDR1 having a sequence set forth as SEQ ID NO: 10, a CDR2 having a sequence set forth as SEQ ID NO: 11 and a CDR3 having a sequence set forth as SEQ ID NO: 12.

In some embodiments, the isolated single-domain antibody according to the invention has the sequence set forth as SEQ ID NO: 13.

In particular, the invention relates to an isolated single-domain antibody (sdAb OptiAlb-11) comprising a CDR1 having at least 80%, preferably at least 90%, more preferably at least 95%, even more preferably at least 99% sequence identity with sequence set forth as SEQ ID NO: 14, a CDR2 having at least 80%, preferably at least 90%, more preferably at least 95%, even more preferably at least 99% sequence identity with sequence set forth as SEQ ID NO: 15 and a CDR3 having at least 80%, preferably at least 90%, more preferably at least 95%, even more preferably at least 99% sequence identity with sequence set forth as SEQ ID NO: 16.

Amino acid sequence identity is preferably determined using a suitable sequence alignment algorithm and default parameters, such as BLAST P (Karlin and Altschul, Proc. Natl Acad. Sci. USA 87(6):2264-2268 (1990)).

In some embodiments, the isolated single-domain antibody according to the invention comprises a CDR1 having a sequence set forth as SEQ ID NO: 14, a CDR2 having a sequence set forth as SEQ ID NO: 15 and a CDR3 having a sequence set forth as SEQ ID NO: 16.

In some embodiments, the isolated single-domain antibody according to the invention has the sequence set forth as SEQ ID NO: 17.

In particular, the invention relates to an isolated single-domain antibody (sdAb OptiAlb-12) comprising a CDR1 having at least 80%, preferably at least 90%, more preferably at least 95%, even more preferably at least 99% sequence identity with sequence set forth as SEQ ID NO: 18, a CDR2 having at least 80%, preferably at least 90%, more preferably at least 95%, even more preferably at least 99% sequence identity with sequence set forth as SEQ ID NO: 19 and a CDR3 having at least 80%, preferably at least 90%, more preferably at least 95%, even more preferably at least 99% sequence identity with sequence set forth as SEQ ID NO: 20.

Amino acid sequence identity is preferably determined using a suitable sequence alignment algorithm and default parameters, such as BLAST P (Karlin and Altschul, Proc. Natl Acad. Sci. USA 87(6): 2264-2268 (1990)).

In some embodiments, the isolated single-domain antibody according to the invention comprises a CDR1 having a sequence set forth as SEQ ID NO: 18, a CDR2 having a sequence set forth as SEQ ID NO: 19 and a CDR3 having a sequence set forth as SEQ ID NO: 20.

In some embodiments, the isolated single-domain antibody according to the invention has the sequence set forth as SEQ ID NO: 21.

It should be further noted that the single-domain antibody anti-albumin as described above cross-react with murine albumin, which is of interest for preclinical evaluation and toxicological studies. It should be noted that the single-domain antibody of the invention exhibits a high affinity to both human and murine albumin (see example 19).

In some embodiments, the single domain antibody is a “humanized” single-domain antibody. As used herein the term “humanized” refers to a single-domain antibody of the invention wherein an amino acid sequence that corresponds to the amino acid sequence of a naturally occurring VHH domain has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional chain antibody from a human being. Methods for humanizing single domain antibodies are well known in the art. Typically, the humanizing substitutions should be chosen such that the resulting humanized single domain antibodies still retain the favorable properties of single-domain antibodies of the invention. The one skilled in the art is able to determine and select suitable humanizing substitutions or suitable combinations of humanizing substitutions.

A further aspect of the invention refers to a cross-competing single-domain antibody which cross-competes for binding albumin with the single-domain antibody of the invention. In some embodiment, the cross-competing single-domain antibody of the present invention cross-competes for binding albumin with the single-domain antibody comprising:
a CDR1 having a sequence set forth as SEQ ID NO: 2, a CDR2 having a sequence set forth as SEQ ID NO: 3 and a CDR3 having a sequence set forth as SEQ ID NO: 4;
a CDR1 having a sequence set forth as SEQ ID NO:6, a CDR2 having a sequence set forth as SEQ ID NO: 7 and a CDR3 having a sequence set forth as SEQ ID NO: 8;
a CDR1 having a sequence set forth as SEQ ID NO:10, a CDR2 having a sequence set forth as SEQ ID NO: 11 and a CDR3 having a sequence set forth as SEQ ID NO: 12;
a CDR1 having a sequence set forth as SEQ ID NO:14, a CDR2 having a sequence set forth as SEQ ID NO: 15 and a CDR3 having a sequence set forth as SEQ ID NO: 16; or
a CDR1 having a sequence set forth as SEQ ID NO:18, a CDR2 having a sequence set forth as SEQ ID NO: 18 and a CDR3 having a sequence set forth as SEQ ID NO: 20.

In some embodiment, the cross-competing single-domain antibody of the present invention cross-competes for binding albumin with the single-domain antibody comprising or consisting in the sequence set forth as SEQ ID NO:5, SEQ ID NO:9, SEQ ID NO:13, SEQ ID NO: 17 or SEQ ID NO:21.

As used herein, the term “cross-competes” refers to single-domain antibodies which share the ability to bind to a specific region of an antigen. In the present disclosure, the single-domain antibody that “cross-competes” has the ability to interfere with the binding of another single-domain antibody for the antigen in a standard competitive binding assay. Such a single-domain antibody may, according to non-limiting theory, bind to the same or a related or nearby (e.g., a structurally similar or spatially proximal) epitope as the single-domain antibody with which it competes. Cross-competition is present if single-domain antibody A reduces binding of single-domain antibody B at least by 60%, specifically at least by 70% and more specifically at least by 80% and vice versa in comparison to the positive control which lacks one of said single-domain antibodies. As the skilled artisan appreciates competition may be assessed in different assay set-ups. One suitable assay involves the use of the Biacore technology (e.g., by using the BIAcore 3000 instrument (Biacore, Uppsala, Sweden)), which can measure the extent of interactions using surface plasmon resonance technology. Another assay for measuring cross-competition uses an ELISA-based approach. Furthermore, a high throughput process for “binning” antibodies based upon their cross-competition is described in International Patent Application No. WO2003/48731.

According to the present invention, the cross-competing antibody as above described retain the activity of the single-antibody comprising or consisting in the sequence set forth as SEQ ID NO:5, SEQ ID NO:9, SEQ ID NO: 13, SEQ ID NO:17 or SEQ ID NO:21.

According to the present invention, the cross-competing antibody as above described exhibit a high affinity to both human and murine albumin.

In some embodiments, the cross-competing antibody as described above and/or the single-domain of the invention bind to a conformational epitope comprising at least the following amino acid sequences: amino acid sequence ranging from the amino acid residue at position 491 to the amino acid residue at position 499 in SEQ ID NO:71, amino acid sequence ranging from the amino acid residue at position 516 to the amino acid residue at position 526 in SEQ ID NO: 71 and amino acid sequence ranging from the amino acid residue at position 559 to the amino acid residue at position 565 in SEQ ID NO:71.

### Chimeric Polypeptides of the Invention:

To validate the effect of single-domain antibody as described above, inventors have generated a bispecific single-domain antibody protein. They have obtained a fusion of single-domain antibody OptiAlb-12 with single-domain antibody KB-VWF-013 (which targets the D′D3-domain of VWF; previously described in patents WO2017129630 and WO2018091621).

This variant was expressed, purified and tested for its capacity to bridge VWF to albumin in vitro.

The bispecific single-domain antibody (named as KB-V13A12) was then used for in vivo studies. Mice having reduced levels of VWF (Von Willebrand disease type-1) received a single dose of the bispecific single-domain antibody. VWF and FVIII levels were followed over 14 days.

These data show that there is a clear increase in VWF and FVIII levels, which is maintained for 10 days.

Thus, in some embodiments, the chimeric polypeptide of the invention may also provide at least one further binding site directed against any desired protein, polypeptide, antigen, antigenic determinant or epitope. Said binding site is directed against to the same protein, polypeptide, antigen, antigenic determinant or epitope for which the single domain antibody of the invention is directed again, or may be directed against a different protein, polypeptide, antigen, antigenic determinant or epitope) from the single domain antibody of the invention.

Typically, the chimeric polypeptide of the invention comprises a single domain antibody of the invention, which is fused at its N terminal end, at its C terminal end, or both at its N terminal end and at its C terminal end to at least one further amino acid sequence, i.e. so as to provide a fusion protein. According to the invention the chimeric polypeptides that comprise a sole single domain antibody are referred to herein as “monovalent” polypeptides. Polypeptides that comprise or essentially consist of two or more single domain antibodies according to the invention are referred to herein as “multivalent” polypeptides.

In some embodiments, the chimeric polypeptide comprises at least one single domain antibody of the invention and at least one other binding unit (i.e. directed against another epitope, antigen, target, protein or polypeptide), which is typically also a single domain antibody. Such a polypeptide is referred to herein as “multispecific” polypeptide; in opposition to a polypeptide comprising the same single domain antibodies (“monospecific” polypeptide).

A “bispecific” polypeptide of the invention is a polypeptide that comprises at least one single domain antibody directed against a first antigen (i.e. albumin) and at least one further binding site directed against a second antigen (i.e. different from albumin), whereas a “trispecific” polypeptide of the invention is a polypeptide that comprises at least one single domain antibody directed against a first antigen (i.e. albumin), at least one further binding site directed against a second antigen (i.e. different from albumin) and at least one further binding site directed against a third antigen (i.e. different from both i.e. first and second antigen); etc.

Accordingly, in a second aspect, the invention relates to a chimeric polypeptide comprising a polypeptide and at least one single-domain antibody directed against albumin.

As used herein, the terms “protein” or “polypeptide” refers to a polymer of two or more of the natural amino acids or non-natural amino acids.

A “fusion” or “chimeric” protein or polypeptide comprises a first amino acid sequence linked to a second amino acid sequence with which it is not naturally linked in nature. The amino acid sequences which normally exist in separate proteins can be brought together in the fusion polypeptide. A fusion protein is created, for example, by chemical synthesis, or by creating and translating a polynucleotide in which the polypeptide regions are encoded in the desired relationship. “Fusion” or “chimeric” polypeptides and proteins includes a combination of a first polypeptide chain, e.g., the single-domain antibody against VWF, with a second polypeptide chain, e.g., a single-domain antibody directed against albumin.

In a particular embodiment, the anti-albumin single-domain antibody according to the invention is linked to another single-domain antibody that recognizes an endogenous plasma protein such as VWF. Such anti-albumin single-domain antibody is called a bispecific single-domain antibody.

Accordingly, in a particular embodiment, the invention relates to a bispecific single-domain antibody comprising the anti-albumin single-domain antibody according to the invention and another single-domain antibody that recognizes an endogenous plasma protein.

In a particular embodiment, the bispecific single-domain antibody according to the invention increases the circulatory half-life of the endogenous plasma protein.

In a particular embodiment, the bispecific single-domain antibody according to the invention increases endogenous plasma levels.

In another embodiment, the anti-albumin single-domain antibody according to the invention is linked to another single-domain antibody that recognizes a polypeptide that is infused in a subject (eg a VWF concentrate).

In a particular embodiment, the bispecific single-domain antibody according to the invention increases the circulatory half-life of the exogenous polypeptide.

In another embodiment, the anti-albumin single-domain antibody according to the invention is combined with a single-domain antibody that targets any polypeptide.

In a particular embodiment, the bispecific single-domain antibody according to the invention increases the circulatory half-life of the single-domain antibody that targets another polypeptide.

In another embodiment, the anti-albumin single-domain antibody is fused to another polypeptide (which is not an sdAb, but eg polypeptide such as VWF).

In a particular embodiment, the bispecific single-domain antibody according to the invention increases the half-life of this polypeptide.

As used herein, the term “half-life” refers to a biological half-life of a particular polypeptide in vivo. Half-life may be represented by the time required for half the quantity administered to a subject to be cleared from the circulation and/or other tissues in the animal. When a clearance curve of a given polypeptide is constructed as a function of time, the curve is usually biphasic with a rapid, α-phase and longer β-phase.

In one embodiment, the chimeric polypeptide comprises any polypeptide, in particular therapeutic polypeptide, preferably having a low blood level or a short half-life leading to (repeated administration to the patient in need thereof. Such therapeutic polypeptide is selected from the group consisting of but not limited to: VWF, FVII, FVIII, FIX, antithrombin, fibrinogen, protein C, or protein S, complement proteins (in particular C2, C9, Mannose-binding Lectin (MBL), C1-inhibitor, factor H-related protein-3), Serpins (in particular SerpinA1, SerpinA3, SerpinA5, SerpinA6, SerpinA7, SerpinA8, SerpinA10, SerpinC1 (=antithrombin), SerpinD1, SerpinE1, SerpinF2, SerpinG1, SerpinI1), fibrinolysis-related proteins (tissue-type plasminogen activator, urokinase-type plasminogen activator, plasminogen), protein-hormones, growth-factors, interleukins, insulin, glucagon, osteoprotegerin (OPG), Angiopoietin-2 (ANGPT2) or furin.

In a particular embodiment, the chimeric polypeptide comprises at least one another isolated single domain antibody.

In a particular embodiment, the chimeric polypeptide comprises a clotting factor (also referred as blood coagulation factor).

As used herein, the term “clotting factor,” refers to molecules, or analogs thereof naturally occurring or recombinant produced which are involved in the process of hemostasis. In other words, it means molecules having pro-clotting activity, i.e., are responsible for the conversion of fibrinogen into a mesh of insoluble fibrin causing the blood to coagulate or clot, or having anti-clotting activity. Pro-clotting factors include factor V, factor VII, factor VIII, Factor IX, factor X, and prothrombin. Anti-clotting factors include protein C, protein S, protein Z, antithrombin, protease nexin-1, tissue factor pathway inhibitor and protein Z-dependent protease inhibitor (ZPI). In a particular embodiment, the chimeric polypeptide according to the invention, wherein the polypeptide is a clotting factor selected from the group consisting of FVII, FVIII, protein C and protein S. Clotting factors of the invention may also be variants of wild-type clotting factors. The term “variants” includes insertions, deletions and substitutions, either conservative or non-conservative, where such changes do not substantially alter the active site, or active domain, which confers the biological activities of the respective clotting factor. Preferably a clotting factor is selected from the group consisting of FVII, FVIII and FX.

In a particular embodiment, the chimeric polypeptide wherein, the at least one another single domain antibody is directed against a clotting factor selected from the group consisting of VWF, FVII, FVIII, antithrombin, fibrinogen, protein C, protein S, complement proteins (in particular C2, C9, Mannose-binding Lectin (MBL), C1-inhibitor, factor H-related protein-3), Serpins (in particular SerpinA1, SerpinA3, SerpinA5, SerpinA6, SerpinA7, SerpinA8, SerpinA10, SerpinC1 (=antithrombin), SerpinD1, SerpinE1, SerpinF2, SerpinG1, SerpinI1), fibrinolysis-related proteins (tissue-type plasminogen activator, urokinase-type plasminogen activator, plasminogen), protein-hormones or growth-factors and interleukins.

In another embodiment, the chimeric polypeptide according to the invention wherein the at least one another single domain antibody is directed against VWF.

The term “VWF” has its general meaning in the art and refers to the human von Willebrand factor (VWF) which is a blood glycoprotein involved in blood clotting. VWF is a monomer composed of several homologous domains each covering different functions: D1-D2-D′-D3-A1-A2-A3-D4-C1-C2-C3-C4-C5-C6-CK. The naturally occurring human VWF protein has an amino acid sequence as shown in GeneBank Accession number NP_000543.2. Monomers are subsequently arranged into dimers or multimers by crosslinking of cysteine residues via disulfide bonds. Multimers of VWF can thus be extremely large and can consist of over 40 monomers also called high molecular weight (HMW)-multimers of VWF.

In some embodiments, the at least one another single domain antibody directed against VWF is as described in WO2017/129630 and WO2018/091621.

In another embodiment, the chimeric polypeptide according to the invention wherein the at least one another single domain directed against VWF comprises:
a CDR1 having a sequence set forth as SEQ ID NO:22, a CDR2 having a sequence set forth as SEQ ID NO: 23 and a CDR3 having a sequence set forth as SEQ ID NO: 24 or
a CDR1 having a sequence set forth as SEQ ID NO:26, a CDR2 having a sequence set forth as SEQ ID NO: 27 and a CDR3 having a sequence set forth as SEQ ID NO: 28.

The sequences of single domain antibody directed against D′D3 domain of VWF (KB-VWF-013 KB and KB-VWF-080) are indicated in the following table (B):

TABLE B
Sequences of sdAb anti-D′D3 VWF domains.

| KB-VWF-013 | |
|---|---|
| domains | Sequences |
| CDR1 | SEQ ID NO: 22 |
| | GRTFIRYAMA |
| CDR2 | SEQ ID NO: 23 |
| | IPQSGGRSYYADSVKG |
| CDR3 | SEQ ID NO: 24 |
| | TSTYYGRSAYSSHSGGYDY |
| SEQUENCE | SEQ ID NO: 25 |
| KB-VWF-013 | QVQLVQSGGGLVQAGDSLRLSCAASGRTFIRYAMAWFRQAPGK |
| | EREFVAAIPQSGGRSYYADSVKGRFTISRDNAKNTVYLQMNSLK |
| | PEDTAVYSCAATSTYYGRSAYSSHSGGYDYWGQGTQVTVSS |
| | (Bold: CDR sequence and not bold: framework sequence) |
| KB-VWF-080 | |
| domains | Sequences |
| CDR1 | SEQ ID NO: 26 |
| | GRTFSDYA |
| CDR2 | SEQ ID NO: 27 |
| | ISRSGGRL |
| CDR3 | SEQ ID NO: 28 |
| | AARTNWNPPRPLPEEYNY |
| SEQUENCE | SEQ ID NO: 29 |
| KB-VWF-080 | QVQLVQSGGGLVQAGGSLKLSCAASGRTFSDYAMGWFRQAPG |
| | KERDFVASISRSGGRLSYADSVKGRFTISRDNAKNTVYLQMNSL |
| | KPEDTAVYYCAARTNWNPPRPLPEEYNYWGQGTQVTVSS |
| | (Bold: CDR sequence and not bold: framework sequence) |

In a particular embodiment, the chimeric polypeptide according to the invention is a bispecific polypeptide.

The chimeric polypeptide according to the invention, wherein the bispecific polypeptide has the following sequence consisting of but not limited to: SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40, SEQ ID NO:47, SEQ ID NO:52, SEQ ID NO:57, SEQ ID NO:62, SEQ ID NO:67, SEQ ID NO:68, SEQ ID NO:69 and SEQ ID NO: 70.

In another embodiment, the chimeric polypeptide according to the invention comprises single-domain antibody anti-albumin and single-domain antibody anti-VWF.

In the context of the invention, the chimeric polypeptide according to the invention is a bispecific polypeptide comprising single-domain antibody anti-albumin and single-domain antibody anti-VWF has the following fusion sequence SEQ ID NO: 30 (KB-V13A12).

In a particular embodiment, the chimeric polypeptide according to the invention comprising single-domain antibody anti-albumin and single-domain antibody anti-VWF has the following fusion sequence SEQ ID NO: 30 (KB-V13A12).

SEQ ID NO: 30:
QVQLVQSGGGLVQAGDSLRLSCAASGRTFIRYAMAWFRQAPGKEREFVAA
IPQSGGRSYYADSVKGRFTISRDNAKNTVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 31 (KB-V13A12/V12L).

SEQ ID NO: 31:
QVQLVQSGGGLLQAGDSLRLSCAASGRTFIRYAMAWFRQAPGKEREFVAA
IPQSGGRSYYADSVKGRFTISRDNAKNTVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 32 (KB-V13A12/E46V).

SEQ ID NO: 32:
QVQLVQSGGGLVQAGDSLRLSCAASGRTFIRYAMAWFRQAPGKERVFVAA
IPQSGGRSYYADSVKGRFTISRDNAKNTVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 33 (KB-V13A12/T78S).

SEQ ID NO: 33:
QVQLVQSGGGLVQAGDSLRLSCAASGRTFIRYAMAWFRQAPGKEREFVAA
IPQSGGRSYYADSVKGRFTISRDNAKNSVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 34 (KB-V13A12/V12L-E46V).

SEQ ID NO: 34:
QVQLVQSGGGLLQAGDSLRLSCAASGRTFIRYAMAWFRQAPGKERVFVAA
IPQSGGRSYYADSVKGRFTISRDNAKNTVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 35 (KB-V13A12/V12L-T78S).

SEQ ID NO: 35:
QVQLVQSGGGLLQAGDSLRLSCAASGRTFIRYAMAWFRQAPGKEREFVAA
IPQSGGRSYYADSVKGRFTISRDNAKNSVYLOMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 36 (KB-V13A12/E46V-T78S).

SEQ ID NO: 36:
QVQLVQSGGGLVQAGDSLRLSCAASGRTFIRYAMAWFRQAPGKERVFVAA
IPQSGGRSYYADSVKGRFTISRDNAKNSVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 37 (KB-V13A12/V12L-E46V-T78S).

SEQ ID NO: 37:
QVQLVQSGGGLLQAGDSLRLSCAASGRTFIRYAMAWFRQAPGKERVFVAA
IPQSGGRSYYADSVKGRFTISRDNAKNSVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 43 (KB-V13A12/V5L).

SEQ ID NO: 43:
QVQLLQSGGGLVQAGDSLRLSCAASGRTFIRYAMAWFRQAPGKEREFVAA
IPQSGGRSYYADSVKGRFTISRDNAKNTVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 44 (KB-V13A12/V5L-E46V).

SEQ ID NO: 44:
QVQLLQSGGGLVQAGDSLRLSCAASGRTFIRYAMAWFRQAPGKERVFVAA
IPQSGGRSYYADSVKGRFTISRDNAKNTVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 45 (KB-V13A12/V5L-T78S).

SEQ ID NO: 45:
QVQLLQSGGGLVQAGDSLRLSCAASGRTFIRYAMAWFRQAPGKEREFVAA
IPQSGGRSYYADSVKGRFTISRDNAKNSVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 46 (KB-V13A12/V5L-E46V-T78S).

SEQ ID NO: 46:
QVQLLQSGGGLVQAGDSLRLSCAASGRTFIRYAMAWFRQAPGKERVFVAA
IPQSGGRSYYADSVKGRFTISRDNAKNSVYLQMNSLKPEDTAVYSCAATS
TYYGRSAYSSHSGGYDYWGQGTQVTVSSAAAGGGSGGGSGGGSGGGSEVQ
LVESGGGLVQPGNSLRLSCAASGFTFSRFGMSWVRQAPGKGLEWVSSISG
SGSDTLYADSVKGRFTISRDNAKTTLYLQMNSLRPEDTAVYYCTIGGSLS
PSSQGTLVTVSS.

In a particular embodiment, the chimeric polypeptide according to the invention comprising single-domain antibody anti-albumin and single-domain antibody anti-VWF has the following fusion sequence SEQ ID NO: 47 (KB-V80A12).

SEQ ID NO: 47:
QVQLVQSGGGLVQAGGSLKLSCAASGRTFSDYAMGWFRQAPGKERDFVAS
ISRSGGRLSYADSVKGRFTISRDNAKNTVYLQMNSLKPEDTAVYYCAART
NWNPPRPLPEEYNYWGQGTQVTVSSAAAGGGSGGGSGGGSGGGSEVQLVE
SGGGLVQPGNSLRLSCAASGFTFSRFGMSWVRQAPGKGLEWVSSISGSGS
DTLYADSVKGRFTISRDNAKTTLYLQMNSLRPEDTAVYYCTIGGSLSPSS
QGTLVTVSS.

In a particular embodiment, the chimeric polypeptide according to the invention comprising single-domain antibody anti-albumin and single-domain antibody anti-VWF, wherein the single-domain antibody anti-VWF is directed against CK domain of VWF. The sequences of single domain antibody directed against CK domain of VWF (KB-VWF-040) are indicated in the following table (C): In a particular embodiment, the sequence of single domain antibody directed against CK domain of VWF (KB-VWF-040) is indicated in the following table (C):

TABLE C
Sequences of sdAb anti-CK domain of VWF.

| KB-VWF-040 | Sequences |
|---|---|
| CDR1 | SEQ ID NO: 48 |
| | GRTFSSNA |
| CDR2 | SEQ ID NO: 49 |
| | AAISWMST |
| CDR3 | SEQ ID NO: 50 |
| | ARREDRRVLTTDYDY |
| SEQUENCE | SEQ ID NO: 51 |
| KB-VWF-040 | QVQLVQSGGGLVQAGGSLRLSCAASGRTFSSNAMAWFRQAPGK |
| | EREFVAAISWMSTTYADSVAGRFTISRDNAKNTVYLQMNSLKPE |
| | DTAVYYCAARREDRRVLTTDYDYWGQGTQVTVSS |
| | (Bold: CDR sequence and not bold: framework sequence) |

In a particular embodiment, the chimeric polypeptide according to the invention comprising single-domain antibody anti-albumin and single-domain antibody anti-VWF has the following fusion sequence SEQ ID NO: 52 (KB-VWF-040).

SEQ ID NO: 52
QVQLVQSGGGLVQAGGSLRLSCAASGRTFSSNAMAWFRQAPGKEREFVAA
ISWMSTTYADSVAGRFTISRDNAKNTVYLQMNSLKPEDTAVYYCAARRED
RRVLTTDYDYWGQGTQVTVSSAAAGGGSGGGSGGGSGGGSEVQLVESGGG
LVQPGNSLRLSCAASGFTFSRFGMSWVRQAPGKGLEWVSSISGSGSDTLY
ADSVKGRFTISRDNAKTTLYLQMNSLRPEDTAVYYCTIGGSLSPSSQGTL
VTVSS

In another embodiment, the chimeric polypeptide according to the invention wherein the at least one another single domain antibody is directed against protein S (PS).

As used herein, the term “Protein S” (PS) refers to a natural anticoagulant acting as a cofactor for activated protein C (APC) in the proteolytic inactivation of activated factor V (FVa) and VIII (FVIIIa), but also for tissue factor pathway inhibitor α (TFPIα) in the inhibition of activated factor X (FXa).

In another embodiment, the chimeric polypeptide according to the invention comprises single-domain antibody anti-albumin and single-domain antibody anti-protein S.

In some embodiments, the at least one another single domain antibody directed against VWF is as described in WO2022/002880.

In a particular embodiment, the chimeric polypeptide according to the invention comprising single-domain antibody anti-albumin and single-domain antibody anti-protein S has the following fusion sequence SEQ ID NO: 38.

SEQ ID NO: 38:
QVQLQESGGGLVQAGGSLRLSCAASGRTFSSYAMGWVRQAPGKEREFVAA
ISYNGGRTNYADSVKGRFTISRDNAKNTGYLQMNSLKPEDTAVYYCAANP
RMWGSVDFRSWGQGTQVTVSSAAAGGGSGGGSGGGSGGGSEVQLVESGGG
LVQPGNSLRLSCAASGFTFSRFGMSWVRQAPGKGLEWVSSISGSGSDTLY
ADSVKGRFTISRDNAKTTLYLQMNSLRPEDTAVYYCTIGGSLSPSSQGTL
VTVSS

In a particular embodiment, the chimeric polypeptide according to the invention comprising single-domain antibody anti-albumin and two single-domain antibody anti-protein S has the following fusion sequence SEQ ID NO: 39.

SEQ ID NO: 39:
QVQLQESGGGLVQAGGSLRLSCAASGRTFSSYAMGWVRQAPGKEREFVAA
ISYNGGRTNYADSVKGRFTISRDNAKNTGYLQMNSLKPEDTAVYYCAANP
RMWGSVDFRSWGQGTQVTVSSGGGSGGGSGGGSGGGSQVQLQESGGGLVQ
AGGSLRLSCAASGRTESSYAMGWVRQAPGKEREFVAAISYNGGRTNYADS
VKGRFTISRDNAKNTGYLQMNSLKPEDTAVYYCAANPRMWGSVDFRSWGQ
GTQVTVSSAAAGGGSGGGSGGGSGGGSEVQLVESGGGLVQPGNSLRLSCA
ASGFTFSRFGMSWVRQAPGKGLEWVSSISGSGSDTLYADSVKGRFTISRD
NAKTTLYLQMNSLRPEDTAVYYCTIGGSLSPSSQGTLVTVSS.

In another embodiment, the chimeric polypeptide according to the invention wherein the at least one another single domain antibody is directed against protease nexin-1 (PN-1).

As used herein, the term “protease nexin-1” (PN-1) also known as SERPINE2, is a member of serine protease inhibitors, termed serpins that are key regulators in many biologic events. PN-1 is a serpin that is barely detectable in plasma but found in many organs and produced by most cell types, including monocytes, platelets, and vascular cells. PN-1 is a 45- to 50-kDa glycoprotein that is encoded by the SERPINE2 gene on human chromosome 2q33-q35. PN-1 is a 378 amino acid residue single-chain containing 3 cysteine residues that do not form disulfide bonds within the protein core of the molecule (Bouton et al., 2012 and Mc Grogan et al 1988 Boulaftali et al., 2010).

In some embodiments, the at least one another single domain antibody directed against protease nexin-1 is as described in WO2020/54619.

In a particular embodiment, the chimeric polypeptide according to the invention comprising single-domain antibody anti-albumin and single-domain antibody anti-protease nexin-1 has the following fusion sequence SEQ ID NO: 40.

SEQ ID NO: 40:
EVQLQASGGGFVQPGGSLRLSCAASGSTWFREIMGWFRQAPGKEREFVSA
ISSDPTWHAYYADSVKGRFTISRDNSKNTVYLQMNSLRAEDTATYYCAPL
AGTESIHWWDPWHESSYWGQGTQVTVSSGGGSGGGSGGGSGGGSEVQLQA
SGGGFVQPGGSLRLSCAASGDTWSLEIMGWFRQAPGKEREFVSAISSEDG
WHAYYADSVKGRFTISRDNSKNTVYLQMNSLRAEDTATYYCAKIENWIQA
VEGEMSDYWGQGTQVTVSSAAAGGGSGGGSGGGSGGGSEVQLVESGGGLV
QPGNSLRLSCAASGFTFSRFGMSWVRQAPGKGLEWVSSISGSGSDTLYAD
SVKGRFTISRDNAKTTLYLQMNSLRPEDTAVYYCTIGGSLSPSSQGTLVT
VSS.

In another embodiment, the chimeric polypeptide according to the invention wherein the at least one another single domain antibody is directed against antithrombin.

As used herein, the term “Antithrombin”, also known as SERPIN C1, has its general meaning in the art and refers to a small glycoprotein that inactivates several enzymes of the coagulation system. Antithrombin activity is increased by anticoagulant heparin, which enhances the binding of antithrombin to factor IIa and factor Xa.

In another embodiment, the chimeric polypeptide according to the invention comprises single-domain antibody anti-albumin and single-domain antibody anti-antithrombin.

In some embodiments, the at least one another single domain antibody directed against antithrombin (KB-AT-01) are indicated in the following table (Table D):

TABLE D
Sequences of sdAb anti-antithrombin.

| KB-AT-01 | Sequences |
|---|---|
| CDR1 | SEQ ID NO: 53 |
| | GRTFRNYV |
| CDR2 | SEQ ID NO: 54 |
| | INRSGAIT |
| CDR3 | SEQ ID NO: 55 |
| | AAGETTWSIRRDDYDY |
| SEQUENCE | SEQ ID NO: 56 |
| KB-AT-01 | QVQLVQSGGGLVQAGGSLRLSCAASGRTFRNYVMGWFRQAPG |
| | KDPEFIAGINRSGAITYYGDSVKGRFTISRDNAKNTVSLQMNSLE |
| | PEDTAVYYCAAGETTWSIRRDDYDYWGQGTQVTVSS |
| | (Bold: CDR sequence and not bold: framework sequence) |

In a particular embodiment, the chimeric polypeptide according to the invention wherein the at least one another single domain directed against antithrombin comprises a CDR1 having a sequence set forth as SEQ ID NO:53, a CDR2 having a sequence set forth as SEQ ID NO: 54 and a CDR3 having a sequence set forth as SEQ ID NO: 55.

In a particular embodiment, the chimeric polypeptide according to the invention comprising single-domain antibody anti-albumin and single-domain antibody anti-antithrombin has the following fusion sequence SEQ ID NO: 57 (KB-AT01A12).

SEQ ID NO: 57
QVQLVQSGGGLVQAGGSLRLSCAASGRTFRNYVMGWFRQAPGKDPEFIAG
INRSGAITYYGDSVKGRFTISRDNAKNTVSLQMNSLEPEDTAVYYCAAGE
TTWSIRRDDYDYWGQGTQVTVSSAAAGGGSGGGSGGGSGGGSEVQLVESG
GGLVQPGNSLRLSCAASGFTFSRFGMSWVRQAPGKGLEWVSSISGSGSDT
LYADSVKGRFTISRDNAKTTLYLQMNSLRPEDTAVYYCTIGGSLSPSSQG
TLVTVSS

In another embodiment, the chimeric polypeptide according to the invention wherein the at least one another single domain antibody is directed against coagulation factor X.

As used herein, the term “coagulation factor X” or “factor X”, has its general meaning in the art and refers to a secreted serine protease implicated in coagulation mechanisms. It serves as the first enzyme in the coagulation cascade to form fibrin. While Factor X normally circulates in the plasma as inactive molecules, the activation of Factor X is involved in both the intrinsic and extrinsic coagulation pathways.

In another embodiment, the chimeric polypeptide according to the invention comprises single-domain antibody anti-albumin and single-domain antibody anti-factor X.

In some embodiments, the at least one another single domain antibody directed against factor X (KB-FX-E3) are indicated in the following table (Table E):

TABLE E
Sequences of sdAb anti-factor X.

| KB-FX-E3 | Sequences |
|---|---|
| CDR1 | SEQ ID NO: 58 |
| | GSISRGDL |
| CDR2 | SEQ ID NO: 59 |
| | ITPGANT |
| CDR3 | SEQ ID NO: 60 |
| | AAASGKGPGRGRKHKY |
| SEQUENCE | SEQ ID NO: 61 |
| KB-FX-E3 | QVQLQESGGGLVQAGGSLRLSCAASGSISRGDLMAWFRQAPGK |
| | ERELVATITPGANTYYADSVKGRFTISRDNTKNTMYLQMNSLKP |
| | EDTAVYFCAAASGKGPGRGRKHKYWGQGTQVTVSS |
| | (Bold: CDR sequence and not bold: framework sequence) |

In a particular embodiment, the chimeric polypeptide according to the invention wherein the at least one another single domain directed against factor X comprises a CDR1 having a sequence set forth as SEQ ID NO:58, a CDR2 having a sequence set forth as SEQ ID NO: 59 and a CDR3 having a sequence set forth as SEQ ID NO: 60.

In a particular embodiment, the chimeric polypeptide according to the invention comprising single-domain antibody anti-albumin and single-domain antibody anti-factor X has the following fusion sequence SEQ ID NO: 62 (KB-X3A12).

SEQ ID NO: 62
QVQLQESGGGLVQAGGSLRLSCAASGSISRGDLMAWFRQAPGKERELVAT
ITPGANTYYADSVKGRFTISRDNTKNTMYLQMNSLKPEDTAVYFCAAASG
KGPGRGRKHKYWGQGTQVTVSSAAAGGGSGGGSGGGSGGGSEVQLVESGG
GLVQPGNSLRLSCAASGFTFSRFGMSWVRQAPGKGLEWVSSISGSGSDTL
YADSVKGRFTISRDNAKTTLYLQMNSLRPEDTAVYYCTIGGSLSPSSQGT
LVTVSS

In another embodiment, the chimeric polypeptide according to the invention wherein the at least one another single domain antibody is directed against coagulation factor IX.

As used herein, the term “coagulation factor IX” or “factor IX”, has its general meaning in the art and refers to a blood clotting factor, a zymogen of serine protease. Upon activation, FIX is converted into the active serine protease and, in the presence of Ca²⁺ and membrane phospholipids, it hydrolyses one arginine-isoleucine bond in factor X to form the activated factor X.

In another embodiment, the chimeric polypeptide according to the invention comprises single-domain antibody anti-albumin and single-domain antibody anti-factor IX.

In some embodiments, the at least one another single domain antibody directed against factor IX (KB-FIX-D9) are indicated in the following table (Table F):

TABLE F
Sequences of sdAb anti-factor IX.

| KB-FIX-D9 | Sequences |
|---|---|
| CDR1 | SEQ ID NO: 63 |
| | GLIFSFNA |
| CDR2 | SEQ ID NO: 64 |
| | ITSGGST |
| CDR3 | SEQ ID NO: 65 |
| | RSSQSGVEY |
| SEQUENCE | SEQ ID NO: 66 |
| KB-FIX-D9 | QVQLVQSGGGLVQPGGSLKLSCAASGLIFSFNALGWYRQAPGK |
| | QRELVAHITSGGSTNYADSVKGRFTISRDNVKKTAFLQMNSLKP |
| | EDTAVYYCRSSQSGVEYWGQGTQVTVSS |
| | (Bold: CDR sequence and not bold: framework sequence) |

In a particular embodiment, the chimeric polypeptide according to the invention wherein the at least one another single domain directed against factor IX comprises a CDR1 having a sequence set forth as SEQ ID NO:63, a CDR2 having a sequence set forth as SEQ ID NO: 64 and a CDR3 having a sequence set forth as SEQ ID NO: 65.

In a particular embodiment, the chimeric polypeptide according to the invention comprising single-domain antibody anti-albumin and single-domain antibody anti-factor X has the following fusion sequence SEQ ID NO: 67 (KB-F9D9A12).

SEQ ID NO: 67
QVQLVQSGGGLVQPGGSLKLSCAASGLIFSFNALGWYRQAPGKQRELVAH
ITSGGSTNYADSVKGRFTISRDNVKKTAFLQMNSLKPEDTAVYYCRSSQS
GVEYWGQGTQVTVSSAAAGGGSGGGSGGGSGGGSEVQLVESGGGLVQPGN
SLRLSCAASGFTFSRFGMSWVRQAPGKGLEWVSSISGSGSDTLYADSVKG
RFTISRDNAKTTLYLQMNSLRPEDTAVYYCTIGGSLSPSSQGTLVTVSS

In a particular embodiment, the chimeric polypeptide according to the invention comprising single-domain antibody anti-albumin and single-domain antibody anti-factor X has the following fusion sequence SEQ ID NO: 68 (KB-F9D9A12/LTRAE).

SEQ ID NO: 68
QVQLVQSGGGLVQPGGSLKLSCAASGLIFSFNALGWYRQAPGKQRELVAH
ITSGGSTNYADSVKGRFTISRDNVKKTAFLQMNSLKPEDTAVYYCRSSQS
GVEYWGQGTQVTVSSGGGSVSQTSKLTRAETVFPDVDGGGSEVQLVESGG
GLVQPGNSLRLSCAASGFTFSRFGMSWVRQAPGKGLEWVSSISGSGSDTL
YADSVKGRFTISRDNAKTTLYLQMNSLRPEDTAVYYCTIGGSLSPSSQGT
LVTVSS

In a particular embodiment, the chimeric polypeptide according to the invention comprising single-domain antibody anti-albumin and single-domain antibody anti-factor X has the following fusion sequence SEQ ID NO: 69 (KB-F9D9A12/FTRVV).

SEQ ID NO: 69
QVQLVQSGGGLVQPGGSLKLSCAASGLIFSFNALGWYRQAPGKQRELVAH
ITSGGSTNYADSVKGRFTISRDNVKKTAFLQMNSLKPEDTAVYYCRSSQS
GVEYWGQGTQVTVSSGGGSTQSFNDFTRVVGGEDAKPGGGSEVQLVESGG
GLVQPGNSLRLSCAASGFTFSRFGMSWVRQAPGKGLEWVSSISGSGSDTL
YADSVKGRFTISRDNAKTTLYLQMNSLRPEDTAVYYCTIGGSLSPSSQGT
LVTVSS

In a particular embodiment, the chimeric polypeptide according to the invention comprising single-domain antibody anti-albumin and single-domain antibody anti-factor X has the following fusion sequence SEQ ID NO: 70 (KB-F9D9A12/LTRVV).

SEQ ID NO: 70
QVQLVQSGGGLVQPGGSLKLSCAASGLIFSFNALGWYRQAPGKQRELVAH
ITSGGSTNYADSVKGRFTISRDNVKKTAFLQMNSLKPEDTAVYYCRSSQS
GVEYWGQGTQVTVSSGGGSVSQTSKLTRVVGGEDAKPGGGSEVQLVESGG
GLVQPGNSLRLSCAASGFTFSRFGMSWVRQAPGKGLEWVSSISGSGSDTL
YADSVKGRFTISRDNAKTTLYLQMNSLRPEDTAVYYCTIGGSLSPSSQGT
LVTVSS

In a particular embodiment, the chimeric polypeptide exhibits an increase of blood level of interest protein when administered to a subject, compared to a corresponding polypeptide not linked to said single-domain antibody directed against albumin and administered to said subject.

Typically, the chimeric polypeptide of the invention comprises at least one single-domain antibody of the invention, which is fused at the N terminal end, at the C terminal end, or both at the N terminal end and at the C terminal end of the therapeutic polypeptide, i.e. so as to provide a fusion protein (eventually via at least one further amino acid sequence).

Alternatively, the chimeric polypeptide of the invention comprises at least one single domain antibody of the invention, which is inserted into the therapeutic polypeptide.

The term “inserted into” as used herein refers to the position of a single-domain antibody directed against albumin in a chimeric polypeptide relative to the analogous position in native polypeptide such as mature human VWF polypeptide. The term refers to the characteristics of the chimeric polypeptide relative to native polypeptide, and do not indicate, imply or infer any methods or process by which the chimeric polypeptide was made.

Importantly, to improve exposure of the single-domain antibody in the context of the fusion protein, amino acid linkers may be placed N- or C-terminally of each single-domain antibody sequence. Examples of linkers to use in the context of the invention are (Gly3-Ser)4, (Gly3-Ser), Ser-Gly or (Ala-Ala-Ala).

As used herein, the term “insertion site” refers to a position in a polypeptide, such as a VWF polypeptide, which is immediately upstream of the position at which a heterologous moiety can be inserted. An “insertion site” is specified as a number, the number being the number of the amino acid in said polypeptide to which the insertion site corresponds, which is immediately N-terminal to the position of the insertion.

According to the invention, the polypeptides that comprise a sole single-domain antibody are referred to herein as “monovalent” polypeptides. Polypeptides that comprise or essentially consist of two or more single-domain antibodies according to the invention are referred to herein as “multivalent” polypeptides.

The chimeric polypeptide according to the invention, comprises at least one single-domain antibody of the invention, wherein said single-domain antibody is fused at the N terminal end, at the C terminal end, both at the N terminal end and at the C terminal end of the therapeutic polypeptide or is inserted within the sequence of the therapeutic polypeptide.

In some embodiments, the polypeptides comprise a single domain antibody of the invention that is linked to an immunoglobulin domain. For example, the polypeptides comprise a single domain antibody of the invention that is linked to an Fc portion (such as a human Fc). Said Fc portion may be useful for increasing the half-life and even the production of the single domain antibody of the invention. For example, the Fc portion can bind to serum proteins and thus increases the half-life on the single domain antibody.

In a particular embodiment, the chimeric polypeptide according to the invention, wherein the polypeptide comprises at least one single-domain antibody directed against a first antigen and at least one further binding site directed against a second antigen.

The new single-domain antibodies directed against CK domain of VWF (KB-VWF-040), directed against coagulation factor X (KB-FX-E3), or directed again Factor IX may be of interest in the prevention or treatment of bleeding disorders.

Accordingly, in another aspect, the invention relates to an isolated single-domain antibody (sdAb) directed against CK domain of VWF comprising a CDR1 having a sequence set forth as SEQ ID NO: 48, a CDR2 having a sequence set forth as SEQ ID NO: 49 and a CDR3 having a sequence set forth as SEQ ID NO: 50. In another embodiment, the isolated single-domain antibody directed against CK domain of VWF according to the invention, wherein said single-domain antibody having at least 70% identity with a sequence set forth as SEQ ID NO: 51. In another embodiment, the isolated single-domain antibody directed against CK domain of VWF according to the invention, wherein said single-domain antibody having or comprises a sequence set forth as SEQ ID NO: 51.

Accordingly, in another aspect, the invention relates to an isolated single-domain antibody (sdAb) directed against factor X comprising a CDR1 having a sequence set forth as SEQ ID NO: 58, a CDR2 having a sequence set forth as SEQ ID NO: 59 and a CDR3 having a sequence set forth as SEQ ID NO: 60. In another embodiment, the isolated single-domain antibody directed against factor X according to the invention, wherein said single-domain antibody having at least 70% identity with a sequence set forth as SEQ ID NO: 61. In another embodiment, the isolated single-domain antibody directed against factor X according to the invention, wherein said single-domain antibody having or comprises a sequence set forth as SEQ ID NO: 61.

Accordingly, in another aspect, the invention relates to an isolated single-domain antibody (sdAb) directed against factor IX comprising a CDR1 having a sequence set forth as SEQ ID NO: 63, a CDR2 having a sequence set forth as SEQ ID NO: 64 and a CDR3 having a sequence set forth as SEQ ID NO: 65. In another embodiment, the isolated single-domain antibody directed against factor IX according to the invention, wherein said single-domain antibody having at least 70% identity with a sequence set forth as SEQ ID NO: 66. In another embodiment, the isolated single-domain antibody directed against factor X according to the invention, wherein said single-domain antibody having or comprises a sequence set forth as SEQ ID NO: 66.

The inventions also relates to the isolated single-domain antibody (sdAb) directed against CK domain of VWF according to the invention, isolated single-domain antibody (sdAb) directed against coagulation factor X according to the invention, or isolated single-domain antibody (sdAb) directed against coagulation factor IX according to the invention for use in therapy, and especially in the prevention or treatment of bleeding disorders.

According to the invention, the single domain antibodies and polypeptides of the invention may be produced by conventional automated peptide synthesis methods or by recombinant expression. General principles for designing and making proteins are well known to those of skill in the art.

The single domain antibodies and polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The single domain antibodies and polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a polypeptide of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the single domain antibodies and polypeptides of the invention, it would be necessary to employ vectors comprising polynucleotide molecules for encoding the single domain antibodies and polypeptides of the invention. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (e.g., a single domain antibody). Thus, a promoter nucleotide sequence is operably linked to a given DNA sequence if the promoter nucleotide sequence directs the transcription of the sequence.

In a particular embodiment, the invention relates to a nucleic acid molecule encoding the single domain antibody of the invention and/or a chimeric polypeptide of the invention.

In a particular embodiment, the invention relates to a vector that comprises the nucleic acid of the invention.

In a particular embodiment, the invention relates to host cell which has been transfected, infected or transformed by the nucleic acid of the invention and/or the vector of the invention.

### Chimeric Polypeptide/Albumin Complexes According to the Invention

In another aspect, the invention relates to a chimeric polypeptide/albumin complex wherein the chimeric polypeptide is a chimeric polypeptide of the invention above described and an albumin polypeptide.

In another embodiment, the chimeric polypeptide/albumin complex according to the invention, wherein the chimeric polypeptide comprises another single-domain antibody that recognizes an endogenous plasma protein such as VWF. In a particular embodiment, the chimeric polypeptide/albumin complex according to the invention increases circulatory half-life of the endogenous plasma protein.

In a particular embodiment, the chimeric polypeptide/albumin complex according to the invention increases endogenous plasma levels.

In another embodiment, the chimeric polypeptide/albumin complex according to the invention, wherein the chimeric polypeptide comprises a single-domain antibody that recognizes a polypeptide that is infused in a subject (eg a VWF concentrate).

In a particular embodiment, the chimeric polypeptide/albumin complex according to the invention increases the circulatory half-life of the exogenous polypeptide.

In another embodiment, the chimeric polypeptide/albumin complex according to the invention, wherein the chimeric polypeptide comprises a single-domain antibody that targets any polypeptide.

In a particular embodiment, the chimeric polypeptide/albumin complex according to the invention increases the circulatory half-life of the single-domain antibody that targets another polypeptide.

In another embodiment, the chimeric polypeptide/albumin complex according to the invention, wherein the chimeric polypeptide comprises another polypeptide (which is not an sdAb, but eg polypeptide such as VWF).

In a particular embodiment, chimeric polypeptide/albumin complex according to the invention increases the half-life of this polypeptide.

In a further embodiment, the single-domain antibody against at least one another target is PEGlated (such as rVWF (PEGrVWF)).

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

### Therapeutic Methods and Uses

The single-domain antibody anti-albumin and/or the chimeric polypeptide as described above are suitable to be used in therapeutic methods.

In a third aspect, the invention relates to an isolated single-domain antibody (sdAb) directed against albumin for use as drug.

In another aspect, the invention relates to a chimeric polypeptide comprising a polypeptide and at least one single-domain antibody of the invention for use as drug.

In still another aspect, the invention relates to a chimeric polypeptide/albumin complex of the invention for use as drug.

According to the invention, a single domain antibody of the invention, a chimeric polypeptide of the invention, or a chimeric polypeptide/albumin complex of the invention is administered to ta subject in need thereof with a therapeutically effective amount.

In a particular embodiment the isolated single-domain antibody (sdAb) directed against albumin according to the invention or a chimeric polypeptide according to the invention for use to increase the circulatory half-life of an endogenous plasma protein, an exogenous polypeptide, a single-domain antibody that targets another polypeptide or a polypeptide.

In a particular embodiment, the anti-albumin single-domain antibody, the chimeric polypeptide or the chimeric polypeptide/albumin complex according to the invention for use to increase the circulatory half-life of an endogenous plasma protein.

In a particular embodiment, the anti-albumin single-domain antibody, the chimeric polypeptide or the chimeric polypeptide/albumin complex according to the invention for use to increase the circulatory half-life of an exogenous polypeptide

In a particular embodiment, the anti-albumin single-domain antibody, the chimeric polypeptide or the chimeric polypeptide/albumin complex according to the invention for use to increase the circulatory half-life of a single-domain antibody that targets another polypeptide.

In a particular embodiment, the anti-albumin single-domain antibody, the chimeric polypeptide or the chimeric polypeptide/albumin complex according to the invention for use to increase the half-life of a polypeptide.

In a particular embodiment, the isolated single-domain antibody directed against albumin according to the invention, a chimeric polypeptide comprising a polypeptide and at least one single-domain antibody directed against albumin according to the invention, or the chimeric polypeptide/albumin complex according to the invention for use in a method for preventing or treating bleeding disorders.

In another embodiment, the invention relates to a method of preventing or treating bleeding disorders in a subject in need thereof, comprising administering to said subject a therapeutically effective amount of an anti-albumin single-domain antibody according to the invention, chimeric polypeptide according to the invention or a chimeric polypeptide/albumin complex according to the invention.

In a particular embodiment, the method according to the invention wherein the bleeding disorder is selected from the group consisting of but not limited to: von Willebrand disease, hemophilia A, or hemophilia B, protein C deficiency, protein S deficiency, antithrombin deficiency, factor XI deficiency, C1-esterase inhibitor deficiency, insulin-deficiency, alpha-1-antitrypsin deficiency, complement C2 deficiency or sickle cell disease.

In a particular embodiment, the isolated single-domain antibody directed against albumin according to the invention, a chimeric polypeptide comprising a polypeptide and at least one single-domain antibody directed against albumin according to the invention, or the chimeric polypeptide/albumin complex according to the invention for use in a method for preventing or treating Von Willebrand disease.

In a particular embodiment, the isolated single-domain antibody directed against albumin according to the invention, a chimeric polypeptide comprising a polypeptide and at least one single-domain antibody directed against albumin according to the invention, or the chimeric polypeptide/albumin complex according to the invention for use in a method for preventing or treating a subject having low level of VWF.

In a particular embodiment, an anti-albumin single-domain antibody according to the invention, a chimeric polypeptide according to the invention or a chimeric polypeptide/albumin complex according to the invention for use to increase the blood level of a therapeutic protein.

In a particular embodiment, an anti-albumin single-domain antibody, a chimeric polypeptide or a chimeric polypeptide/albumin complex according to the invention for use to increase the blood level of a clotting factor selected from the group consisting of but not limited to: VWF, FVII, FVIII, antithrombin, fibrinogen, protein C, protein S, complement proteins (in particular C2, C9, Mannose-binding Lectin (MBL), C1-inhibitor, factor H-related protein-3), Serpins (in particular SerpinA1, SerpinA3, SerpinA5, SerpinA6, SerpinA7, SerpinA8, SerpinA10, SerpinC1 (=antithrombin), SerpinD1, SerpinE1, SerpinF2, SerpinG1, SerpinI1), fibrinolysis-related proteins (tissue-type plasminogen activator, urokinase-type plasminogen activator, plasminogen), protein-hormones or growth-factors and interleukins.

In a particular embodiment, the chimeric polypeptide as described above (such as KB-V13A12) for increasing the level of VWF in a subject in need thereof.

In a particular embodiment, the chimeric polypeptide as described above (such as KB-V13A12) for use in a method for treating Von Willebrand disease type-1.

For instance, an anti-albumin single-domain antibody according to the invention, a chimeric polypeptide according to the invention or a chimeric polypeptide/albumin complex according to the invention for use in a method for preventing and/or treating bleeding disorders.

The bleeding disorders that may be treated by administration of the single-domain antibody according to the invention, a chimeric polypeptide according to the invention or a chimeric polypeptide/albumin complex of the invention include, but are not limited to, hemophilia, as well as deficiencies or structural abnormalities in VWF, FVII, FVIII, antithrombin, fibrinogen, protein C, protein S, complement proteins (in particular C2, C9, Mannose-binding Lectin (MBL), C1-inhibitor, factor H-related protein-3), Serpins (in particular SerpinA1, SerpinA3, SerpinA5, SerpinA6, SerpinA7, SerpinA8, SerpinA10, SerpinC1 (=antithrombin), SerpinD1, SerpinE1, SerpinF2, SerpinG1, SerpinI1), fibrinolysis-related proteins (tissue-type plasminogen activator, urokinase-type plasminogen activator, plasminogen), protein-hormones or growth-factors and interleukins.

In a particular embodiment, the bleeding disorder that may be treated by administration of the single-domain antibody, a chimeric polypeptide or a chimeric polypeptide/albumin complex of according to the invention is selected from the group consisting of but not limited to: von Willebrand disease, hemophilia A, or hemophilia B, protein C deficiency, protein S deficiency, antithrombin deficiency, factor XI deficiency, C1-esterase inhibitor deficiency, insulin-deficiency, alpha-1-antitrypsin deficiency, complement C2 deficiency or sickle cell disease.

In a particular embodiment, the bleeding disorder that may be treated by administration of the single-domain antibody, a chimeric polypeptide or a chimeric polypeptide/albumin complex of according to the invention is von Willebrand disease, hemophilia A or hemophilia B.

In a particular embodiment, the bleeding disorder that may be treated by administration of the single-domain antibody, a chimeric polypeptide or a chimeric polypeptide/albumin complex of according to the invention is Von Willebrand disease type-1.

In a particular embodiment, the invention relates to a method of extending or increasing half-life of a therapeutic single-domain antibody against a clotting factor comprising a step of adding to the therapeutic single-domain antibody at least one sdAb directed against albumin.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the terms “administering” or “administration” refer to the act of injecting or otherwise physically delivering a substance as it exists outside the body (e.g., an anti-albumin single-domain antibody or chimeric polypeptide according to the invention) into the subject, such as by oral, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In a particular embodiment, the anti-albumin single-domain antibody or chimeric polypeptide according to the invention is administered orally.

By a “therapeutically effective amount” is meant a sufficient amount of the polypeptide (or the nucleic acid encoding for the polypeptide) to prevent for use in a method for the treatment of acute exacerbation of chronic obstructive pulmonary disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Composition

In a fourth aspect, the invention relates to a pharmaceutical composition comprising a single-domain antibody directed against albumin, a chimeric polypeptide, a chimeric polypeptide/albumin complex as described herein, and a pharmaceutically acceptable carrier.

In a particular embodiment, the pharmaceutical composition according to the invention for use in the prevention or treatment of bleeding disorders as described above.

In a particular embodiment, the pharmaceutical composition according to the invention may include any further agent which is used in the prevention or treatment of bleeding disorders.

In a particular embodiment, the pharmaceutical composition according to the invention for use to increase the circulatory half-life of an endogenous plasma protein.

In a particular embodiment, the pharmaceutical composition according to the invention for use to increase the circulatory half-life of an exogenous polypeptide

In a particular embodiment, the pharmaceutical composition according to the invention for use to increase the circulatory half-life of a single-domain antibody that targets another polypeptide.

In a particular embodiment, the pharmaceutical composition according to the invention for use to increase the half-life of a polypeptide.

In one embodiment, said additional active agents may be contained in the same composition or administrated separately.

In another embodiment, the pharmaceutical composition of the invention relates to combined preparation for simultaneous, separate or sequential use in the prevention and treatment of bleeding disorders.

The single-domain antibodies and polypeptides of the invention (or the nucleic acid encoding thereof) may be combined with pharmaceutically acceptable excipients, and optionally sustained-release matrices, such as biodegradable polymers, to form pharmaceutical compositions. As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The single-domain antibody or the chimeric polypeptide according to the invention (or nucleic acid encoding thereof) may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. The invention will be further illustrated by the following figures and examples.

Finally, the invention also provides kits comprising at least one single domain antibody or chimeric polypeptide of the invention. Kits containing an anti-albumin single domain antibody or chimeric polypeptide of the invention for use in therapeutic methods.

The invention will be further illustrated by the following figures and examples. However, these examples and figures should not be interpreted in any way as limiting the scope of the present invention.

### FIGURES

FIG. 1: Binding of ALB8 to human and mouse serum albumin. Various concentrations of purified ALB were incubated with immobilized human serum albumin (HSA) or mouse serum albumin (MSA). Bound ALB8 was probed using polyclonal peroxidase-labelled anti-cMyc antibodies and detected via hydrolysis of 3,3′,5,5′-tetramethylbenzidine.

FIG. 2: Binding of mutant ALB8 variants to mouse serum albumin. Various concentrations of ALB8 and mutants thereof were incubated with immobilized mouse serum albumin (MSA). Bound nanobodies were probed using polyclonal peroxidase-labelled anti-cMyc antibodies and detected via hydrolysis of 3,3′,5,5′-tetramethylbenzidine.

FIG. 3: Circulatory survival of ALB8 and OptiAlb variants in mice. Factor VIII-deficient mice intravenously received biotinylated ALB8 or OptiAlb variants (2.5 mg/kg), and blood was collected at 5 min, 4 h, 24h or 96h. Residual plasma levels of each biotinylated single-domain antibody were determined. Plotted are residual single-domain antibody levels relative to t=5 min (arbitrarily set at 100%) versus time.

FIG. 4: Binding of OptiAlb-12 and ALB8 to human serum albumin. Various concentrations of ALB8 and OptiAlb-12 thereof were incubated with immobilized human serum albumin (HSA). Bound nanobodies were probed using polyclonal peroxidase-labelled anti-cMyc antibodies and detected via hydrolysis of 3,3′,5,5′-tetramethylbenzidine.

FIG. 5: KB-V13A12 increases VWF and FVIII plasma levels. 129Sv mice expressing human VWF and human GpIbalpha received a single dose of KB-V13A12 (100 microgram/mouse) via a subcutaneous injection. Plasma levels of VWF and FVIII were determined before injection as well as at day 1, 3, 7, 10 and 14 after injection. A sustained statistically significant increase in plasma levels of VWF and FVIII levels over a period of 10 days was observed.

FIG. 6: Correction of hemostasis after injection with KB-V13A12. KB-V13A12 was given subcutaneously to VWD-type 1 mice and three days after injection the terminal tip of the tail was amputated from anesthetized mice. Blood loss was monitored for 30 min. As control, WT mice and untreated VWD-type 1 mice were used. Whereas untreated VWD-type 1 mice have significantly increased blood loss compared to WT mice, VWD-type 1 mice treated with KB-V13A12 has similar blood loss as WT mice.

FIG. 7: In vivo survival of von Willebrand factor in the presence of KB-V13A12. Purified recombinant VWF was given intravenously to VWF-deficient mice (0.5 mg/kg) in the absence or presence of a tenfold molar excess of KB-V13A12. At 24 h after injection, blood was collected and plasma was analysed for the presence of residual VWF antigen. VWF concentrations in mice receiving VWF alone proved 6.6-fold lower compared to mice receiving VWF in the presence of KB-V13A12. Plotted are residual VWF levels (percentage of the amount injected) for each individual mouse included in the study. Apparently, KB-V13A12 protects VWF against rapid removal from the circulation.

FIG. 8: KB-V80A12 differentially increases VWF and FVIII plasma levels. 129Sv mice expressing human VWF and human GpIbalpha received a single dose of KB-V80A12 (100 microgram/mouse) via a subcutaneous injection. Plasma levels of VWF and FVIII were determined before injection as well as at day 3, 6, 10 and 14 after injection. A sustained statistically significant increase in plasma levels of FVIII levels over a period of 10 days was observed, whereas a minor increase in VWF antigen levels were detected.

FIG. 9: Epitope OptiAlb-12. Representation of OptiAlb-12 bound to residues Thr491-Lys499, Glu516-Phe526 and His559-Lys565 of albumin. Three-dimensional structure of OptiAlb-12 was modeled by using the VH-structure available in crystal structure7CJ2 (https://www.rcsb.org/structure/7CJ2) and the albumin structure was obtained from crystal structure 1A06 (https://www.rcsb.org/structure/lao6). Figure was prepared using PyMol.

FIG. 10: Simultaneous binding of KB-V13A12 to VWF and albumin. Wells coated with human or murine albumin were incubated with KB-V13A12 and subsequently with various concentrations of human VWF. Bound VWF was probed using peroxidase-labelled polyclonal anti-VWF antibodies and detected via hydrolysis of 3,3′,5,5′-tetramethylbenzidine. Plotted is response (OD450) versus VWF concentration.

FIG. 11: Binding of humanized KB-V13A12 variants to VWF. Various concentrations of KB-V13A12, KB-V13A12/T78S, KB-V13A12/E46V-T78S, KB-V13A12/V5L-E46V, KB-V13A12/V5L-T78S, KB-V13A12/V5L-E46V-T78S were incubated with immobilized VWF. Bound nanobodies were probed using polyclonal peroxidase-labelled anti-cMyc antibodies and detected via hydrolysis of 3,3′,5,5′-tetramethylbenzidine. Plotted is response (OD450) versus single-domain antibody concentration.

FIG. 12: Binding of KB-V13A12 to albumin and VWF at neutral and low pH. Wells coated with human albumin were incubated with KB-V13A12 and subsequently with various concentrations of human VWF. After three consecutive incubations with 42 mM citric acid/58 mM Na₂HPO₄/150 mM NaCl (pH 5.6) or 9.2 mM citric acid/90.9 mM Na₂HPO₄/150 mM NaCl (pH 7.4) for 20 min at 37° C., bound VWF was probed using peroxidase-labelled polyclonal anti-VWF antibodies and detected via hydrolysis of 3,3′,5,5′-tetramethylbenzidine. Plotted is response (OD450) versus VWF concentration.

FIG. 13: Bridging human albumin and antithrombin using KB-AT01A12. Wells coated with human or murine albumin were incubated with KB-AT01A12 and subsequently with various concentrations of human antithrombin. Bound antithrombin was probed using peroxidase-labelled polyclonal anti-antithrombin antibodies and detected via hydrolysis of 3,3′,5,5′-tetramethylbenzidine. Plotted is response (OD450) versus antithrombin concentration.

FIG. 14: KB-FXE3A12 simultaneously binds HSA and factor X. Wells coated with human or murine albumin were incubated with KB-FXE3A12 and subsequently with various concentrations of human factor X. Bound factor X was probed using peroxidase-labelled polyclonal anti-factor X antibodies and detected via hydrolysis of 3,3′,5,5′-tetramethylbenzidine. Plotted is response (OD450) versus antithrombin concentration.

FIG. 15: KB-F9D9A12 simultaneously binds HSA and factor IX. Wells coated with human albumin were incubated with KB-F9D9A12 and subsequently with various concentrations of human factor IX. Bound factor IX was probed using peroxidase-labelled polyclonal anti-factor IX antibodies and detected via hydrolysis of 3,3′,5,5′-tetramethylbenzidine. Plotted is response (OD450) versus antithrombin concentration.

### EXAMPLES

### Example 1 Expression and Purification of ALB8

The nucleotide sequence encoding ALB8 was cloned into the pHEN6-plasmid, containing a PelB signal peptide for targeting to the periplasmic compartment as well as a C-terminal histidine- and cMyc-tag. The plasmid was used to transform E. Coli WK6 bacteria. E. coli WK6 clones expressing ALB8 were plated and a single clone was used to inoculate 3 ml Luria-Bertani Broth (LB) supplemented with ampicillin. This mixture was incubated for 2.5h at 37° C. under agitation. This pre-culture was then added to 250 ml of prewarmed Terrific Broth (TB) containing 0.1% glucose and 0.1 mg/ml ampicillin, and bacteria were grown under agitation at 37° C. until optical density (OD) 600 nm was between 1.0 and 1.3. Bacteria were collected via centrifugation, and the bacterial pellet was resuspended with 10 ml of TES-buffer (0.2 M Tris (pH8.0), 0.65 mM EDTA, 0.5 M sucrose). After incubation for 1 h at 4° C., 20 ml of 4-fold diluted TES-buffer was added, and the suspension was incubated for 1 h at 4° C. The suspension was then centrifuged and the supernatant was collected into a 50-ml falcon tube. After another centrifugation step, the supernatant was filtered over a 0.22-micron filter.

The filtered protein was next purified via immobilized metal affinity chromatography using a 1-ml HiTrap-Talon column (GE Healthcare) according to the manufacturer's instructions. Eluted protein fractions were analysed via SDS-Page. Purified ALB8 essentially migrated as a single band (data not shown). Fractions 2&3 were pooled and dialyzed against PBS. The protein concentration of the pooled fraction was 7.1 mg/ml, and the volume was 2 ml.

### Example 2 Binding of ALB8 to Human and Mouse Serum Albumin

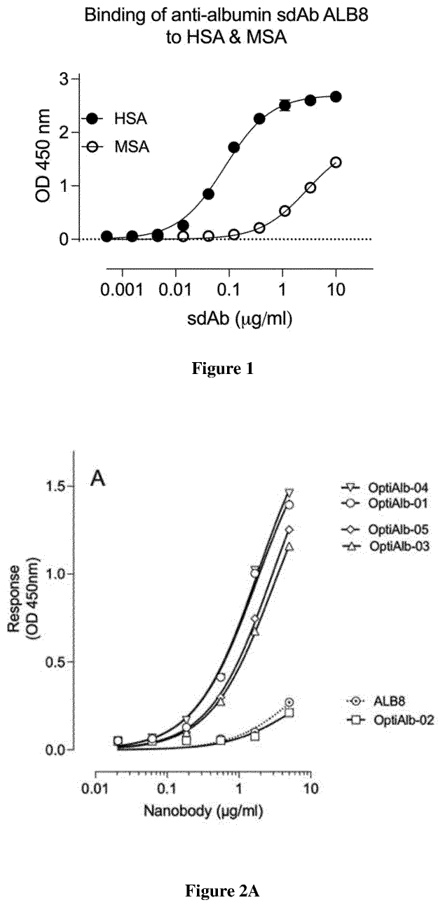

Maxisorb microtiter plates were coated with human serum albumin (HSA) or mouse serum albumin (MSA) in carbonate buffer (pH 9.8) at 10 microgram/ml overnight at 4° C. After washing wells with PBS/0.1% Tween-20, wells were blocked by incubating with PBS/1% bovine serum albumin (BSA) for 1 h at 37° C. Serial dilutions of ALB8 were prepared (0-10 microgram/ml) in PBS/0.1% Tween-20, and preparations were incubated in wells coated with HSA or MSA for 1 h at 37° C. After three washes with PBS/0.1% Tween-20, bound ALB8 was probed using polyclonal peroxidase-labelled anti-cMyc antibodies (1:5,000 dilution), and detected via hydrolysis of 3,3′,5,5′-tetramethylbenzidine. Depicted in FIG. 1 is the response at OD450 nm versus the sdAb concentration. Data were fitted using a model describing one-site binding (GraphPad Prism) to calculate half-maximal binding. Half-maximal binding of ALB8 to HSA was achieved at 0.08 microgram/ml, whereas binding of ALB8 to MSA was achieved at 35-fold higher concentrations (2.86 microgram/ml).

### Example 3 Generation of Randomly Mutated ALB8-Variants

Primers overlapping each of the three CDRs were designed to construct two libraries. In the first library, each amino acid in the CDR1, CDR2 and CDR3 was replaced by one of 19 possible other amino acids (with the exception of cysteine). In the second library, the combined CDR sequences contained one, two or three mutations, with 19 possible amino acid replacements for each amino acid in the three CDRs. The libraries were cloned into the pSTALK-Halo plasmid. These libraries were used to transform yeast, in order to perform yeast-display analysis. With the first library, 703 distinct yeast clones were isolated, whereas with the second library 3.62 million yeast clones were obtained. From each library, 48 random clones were sequenced to validate the diversity of the libraries.

### Example 4 Isolation of Yeast Clones Via Cell Sorting

The surface expression of mutated variants was induced via incubation with galactose. One million yeast cells of the simple variant library were first incubated with Halo-Alexa660 ligand (for the detection of yeast expressing sdAb at the surface) and 100 nM Alexa488-labeled MSA in PBS/2% bovine albumin overnight at 4° C. Cells were then washed in PBS (pH5.6) for 1 h at 30° C. before cell sorting was applied. Fifty thousand (50,000) yeast cells positive for both Halo-Alexa660 and MSA-Alexa488 were sorted using the FACs ARIA III (Becton Dickonson). The sorted cells were amplified and one million cells were subsequently incubated with Halo-Alexa660 and 10 nM MSA-Alexa488 for overnight at 4° C. After washing in PBS (pH5.6), a second round of sorting was performed, and five thousand (5,000) yeast cells were sorted. This second procedure was repeated once, which allowed for the sorting of fifteen thousand (15,000) clones positive for both Halo-Alexa660 and 10 nM MSA-Alexa488. 96 clones were randomly picked and analysed for their sequence, revealing that 11 different mutant clones were isolated.

With regard to the second library, a similar approach was used: eleven million yeast cells were incubated with Halo-Alexa660 and 100 nM MSA-Alexa488 in PBS/2% bovine albumin overnight at 4° C. Cells were then washed in PBS (pH5.6) for 1 h at 30° C. before cell sorting was applied. Five hundred thousand (500,000) yeast cells positive for both Halo-Alexa660 and MSA-Alexa488 were sorted using the FACs ARIA III (Becton Dickinson). The sorted cells were amplified and five million cells were subsequently incubated with Halo-Alexa660 and 10 nM MSA-Alexa488 for overnight at 4° C. After washing in a buffer at pH5.6, a second round of sorting was performed. Three thousand (3,000) clones were sorted and amplified for a third round. In this third round, one million yeast cells were incubated with 10 nM MSA-Alexa4488 and Halo-Alexa660 ligand as described above, and another one million yeast cells were incubated with 1 nM MSA-Alexa4488 and Halo-Alexa660 ligand. Twenty-five thousand (25,000) clones were sorted from the 10 nM MSA-Alexa488 incubation and five thousand (5,000) for the 1 nM MSA-Alexa488 condition. 96 clones were picked from each condition for sequence analysis, identifying 18 different mutant clones from the 10 nM MSA-Alexa488 condition and 17 clones from the 1 nM MSA-Alexa488 condition.

In total, 11 plus 18 plus 17=46 mutant clones were identified, and fourteen unique sequences were obtained.

### Example 5 Binding of Mutant ALB8 Variants to Mouse Serum Albumin

Fourteen mutant ALB8 variants (designated OptiAlb-01 to OptiAlb-14) were cloned into pHEN6 plasmid and produced and purified as described in example 1. Concentrations of the nanobodies were as follows:

TABLE 1

| ALB8 | Concentration |
|---|---|
| variant | (mg/ml) |
| OptiAlb-01 | 4.3 |
| OptiAlb-02 | 3.2 |
| OptiAlb-03 | 2.4 |
| OptiAlb-04 | 1.7 |
| OptiAlb-05 | 3.6 |
| OptiAlb-06 | 1.5 |
| OptiAlb-07 | 3.0 |
| OptiAlb-08 | 0.9 |
| OptiAlb-09 | 3.3 |
| OptiAlb-10 | 3.8 |
| OptiAlb-11 | 2.6 |
| OptiAlb-12 | 3.2 |
| OptiAlb-13 | 3.4 |
| OptiAlb-14 | 4.0 |

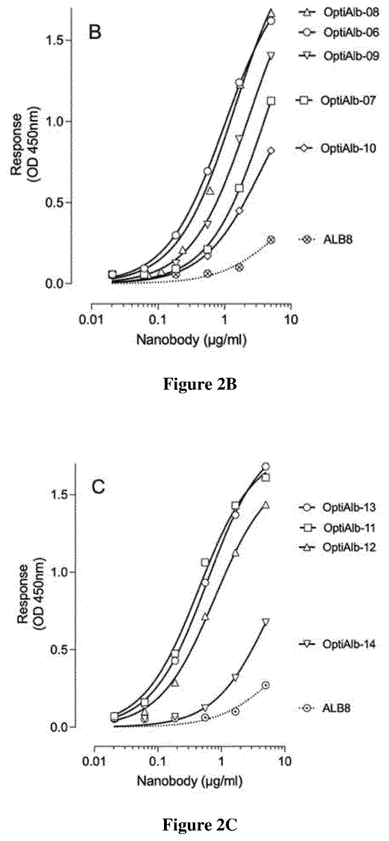

A maxisorb microtiter plate was coated with mouse serum albumin (2 microgram/ml) in carbonate buffer (pH 9.8) overnight at 4° C. After washing wells with PBS/0.1% Tween-20, wells were blocked by incubating with PBS/1% bovine serum albumin (BSA) for 1 h at 37° C. Serial dilutions of ALB8 and the OptiAlb variants were prepared (0-5 microgram/ml) in PBS/0.1% Tween-20, and preparations were incubated in wells coated with MSA for 1 h at 37° C. After three washes with PBS/0.1% Tween-20, bound proteins were probed using polyclonal peroxidase-labelled anti-cMyc antibodies (1:5,000 dilution), and detected via hydrolysis of 3,3′,5,5′-tetramethylbenzidine. We then plotted response (OD450 nm) versus single-domain antibody concentration (OptiAlb-01 to OptiAlb-05, FIG. 2A; OptiAlb-06-OptiAlb-10 FIG. 2B, OptiAlb-11 to OptiAlb-14 FIG. 2C; ALB8 is presented in each panel of FIG. 2).

As depicted in FIG. 2, all mutated ALB8 with the exception of OptiAlb-02 displayed improved binding to MSA.

### Example 6 Circulatory Survival of ALB8 and OptiAlb Variants in Mice

ALB8 and OptiAlb variants were biotinylated using EZ-link NHS-PEG4-biotin (ThermoFisher) according to the manufacturer instructions. Final protein concentrations were:

TABLE 2

| ALB8 | Concentration |
|---|---|
| variant | (mg/ml) |
| OptiAlb-01 | 1.2 |
| OptiAlb-02 | 1.2 |
| OptiAlb-03 | 1.3 |
| OptiAlb-04 | 1.1 |
| OptiAlb-05 | 0.9 |
| OptiAlb-06 | 1.2 |
| OptiAlb-07 | 1.1 |
| OptiAlb-08 | 1.1 |
| OptiAlb-09 | 1.0 |
| OptiAlb-10 | 1.2 |
| OptiAlb-11 | 1.1 |
| OptiAlb-12 | 1.0 |
| OptiAlb-13 | 1.1 |
| OptiAlb-14 | 1.1 |
| ALB8 | 1.1 |

FVIII-deficient mice (bred on a C57B6 background) received biotinylated-ALB8 or biotinylated-OptiAlb variants at a dose of 2.5 mg/kg via intravenous injection into the retro-orbital sinus. Blood was collected at 5 min, 4 h, 24 h and 96h after injection. Plasma samples were prepared via centrifugation (1,500 g for 20 min at room temperature), and analysed for the presence of biotinylated single-domain antibody.

Protein concentrations were determined as follows. 96-well MaxiSorp microtiter plates were coated with 50 microliter of a 5 microgram/ml streptavidin solution in carbonate buffer (pH 9.8) overnight at 4° C. After washing with PBS/0.1% Tween-20, wells were blocked with PBS/1% bovine serum albumin (BSA) for 1 h at 37° C. Wells were then washed with PBS/0.1% Tween-20. Serial dilutions (1/500-1/8000) of plasma samples prepared in PBS/0.1% Tween-20. As reference, serial dilutions of purified biotinylated ALB8 were used. Samples were incubated with immobilized streptavidin for 1 h at 37° C. Well were then washed using PBS/0.1% Tween-20. Bound biotinylated nanobodies were probed using polyclonal peroxidase-labelled anti-cMyc antibodies and detected via hydrolysis of 3,3′,5,5′-tetramethylbenzidine.

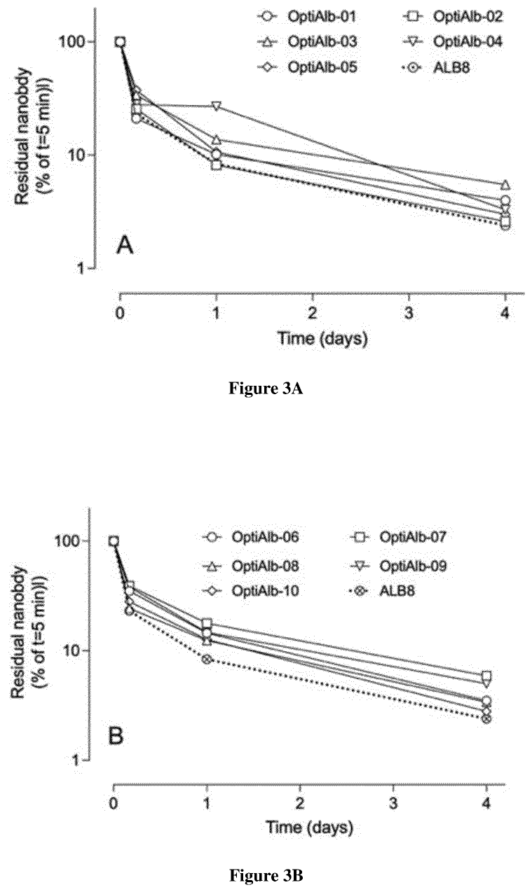

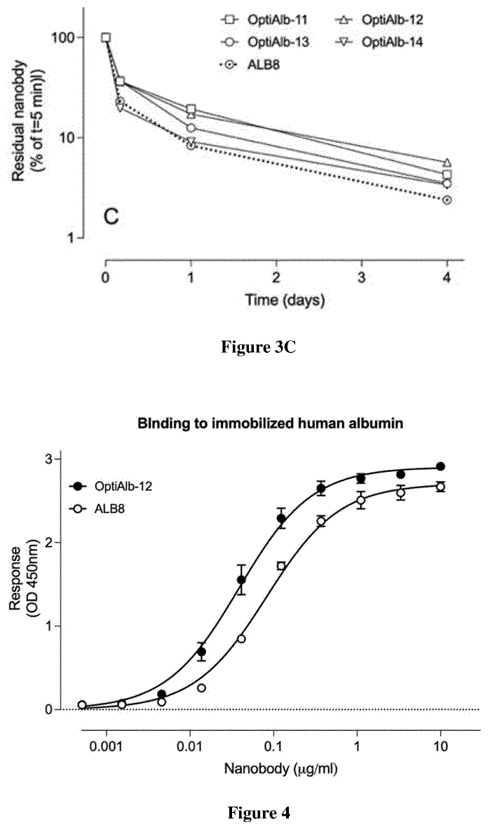

For each single-domain antibody, the residual plasma concentrations were calculated and normalized for the plasma concentration at 5 min after injection. These data were then plotted against time after injection (see FIG. 3). In table 2, relative plasma levels at 96h (as percentage of levels at t=5 min) are summarized. In addition, this table summarizes the ratio of the relative plasma levels for each OptiAlb variant over ALB8. Five OptiAlb variants displayed at least 1.8-fold higher plasma levels at 96h compared to ALB8: OptiAlb-03 (2.3-fold), OptiAlb-07 (2.4-fold), OptiAlb-09 (2.1-fold), OptiAlb-11 (1.8-fold) and OptiAlb-12 (2.4-fold). OptiAlb-03 is having SEQ ID NO:5, OptiAlb-07 is having SEQ ID NO:9, OptiAlb-09 is having SEQ ID NO:13, OptiAlb-11 is having SEQ ID NO:17 and OptiAlb-12 is having SEQ ID NO:21.

TABLE 3

| | Relative plasma levels | Ratio plasma |
|---|---|---|
| ALB8 | at 96 h compared to | levels at 96 h |
| variant | 5 min (in %) | OptiAlb/ALB8 |
| OptiAlb-01 | 4.0 | 1.7 |
| OptiAlb-02 | 2.6 | 1.1 |
| OptiAlb-03 | 5.5 | 2.3 |
| OptiAlb-04 | 3.3 | 1.4 |
| OptiAlb-05 | 3.0 | 1.3 |
| OptiAlb-06 | 3.5 | 1.5 |
| OptiAlb-07 | 5.8 | 2.4 |
| OptiAlb-08 | 3.4 | 1.4 |
| OptiAlb-09 | 5.0 | 2.1 |
| OptiAlb-10 | 2.8 | 1.2 |
| OptiAlb-11 | 4.3 | 1.8 |
| OptiAlb-12 | 5.7 | 2.4 |
| OptiAlb-13 | 3.5 | 1.5 |
| OptiAlb-14 | 3.4 | 1.4 |
| ALB8 | 2.4 | 1.0 |

### Example 7 Binding of OptiAlb-12 and ALB8 to Human Serum Albumin

OptiAlb-12 and ALB8 were compared for binding to immobilized human serum albumin (HSA). A MaxiSorb microtiter plate was coated with HSA (5 microgram/ml) in carbonate buffer (pH 9.8) overnight at 4° C. After washing wells with PBS/0.1% Tween-20, wells were blocked by incubating with PBS/1% bovine serum albumin (BSA) for 1 h at 37° C. Serial dilutions of ALB8 and OptiAlb-12 were prepared (0-10 microgram/ml) in PBS/0.1% Tween-20, and preparations were incubated in wells coated with HSA for 1 h at 37° C. After three washes with PBS/0.1% Tween-20, bound proteins were probed using polyclonal peroxidase-labelled anti-cMyc antibodies (1:5,000 dilution), and detected via hydrolysis of 3,3′,5,5′-tetramethylbenzidine. Both ALB8 and OptiAlb-12 displayed a dose-dependent binding to immobilized HSA (FIG. 4). Data were fitted using a model describing one-site binding (GraphPad Prism) to calculate half-maximal binding. Half-maximal binding of ALB8 to HSA was achieved at 0.083 microgram/ml (95%-confidence interval 0.073-0.094). Half-maximal binding of OptiAlb-12 to HSA was achieved at 0.038 microgram/ml (95%-confidence interval 0.034-0.044), a concentration 2.2-fold lower compared to ALB8 (p<0.0001). OptiAlb-12 (identified by SEQ ID NO:21) thus binds more efficiently than ALB8 to both human and mouse serum albumin, and displays a longer circulatory survival in mice.

### Example 8 KB-V13A12 Increases VWF and FVIII Plasma Levels

A construct encoding KB-VWF-013 fused to OptiAlb-12 (SEQID NO: 30) was cloned into the pHEN6-plasmid so to comprise a C-terminal histidine and cMyc-tag. The bispecific single-domain antibody was expressed and purified as described in Example 1. The purified protein was at a concentration of 5.96 mg/ml, and is designated as KB-V13A12.

Control immunosorbent assays demonstrated that KB-V13A12 can simultaneously bind to both albumin and VWF.

The purified protein was then used for in vivo studies. Transgenic 129Sv mice expressing human von Willebrand factor (VWF) and human Glycoprotein Ibalpha were used for this study. VWF levels in these mice are 15±4% compared to normal human plasma and factor VIII (FVIII) activity levels are 44±8% compared to normal human plasma. This mouse model thus represents a model for von Willebrand disease type 1.

These mice received 100 microgram of KB-V13A12 via subcutaneous injection. Blood samples were taken 24h before injection, and plasma levels of VWF and FVIII from these samples were defined as t=0. Additional blood samples were taken at day 1, day 3, day 7, day 10 and day 14. Plasma samples were prepared and analysed for levels of VWF antigen and FVIII activity.

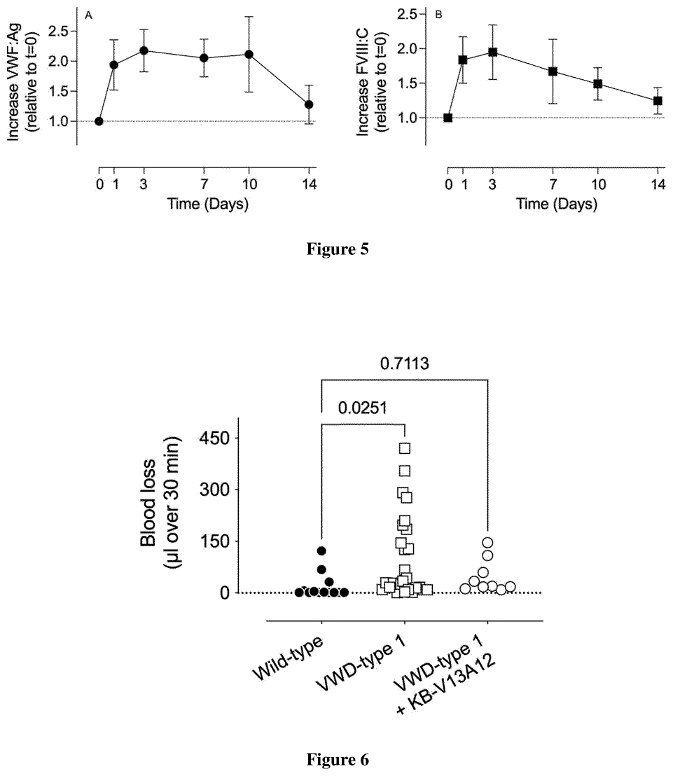

Relative changes in plasma levels of VWF and FVIII are depicted in FIGS. 5A and 5B, respectively. Plasma levels of VWF increased between 1.9±0.4 fold (n=9; p<0.0001 compared to t=0) at day 1 and 2.1±0.6 fold (n=5; p<0.0001 compared to t=0) at day 10. At day 14, plasma levels were similar to that of t=0 (1.3±0.3 fold increase; p=0.526).

As for FVIII activity, plasma levels increased 1.8±0.3 fold (n=9, p<0.0001 compared to t=0) at day 1 and 1.5±0.2 fold (n=5; p=0.038 compared to t=0) at day 10. At day 14, plasma levels were similar to that of t=0 (1.2±0.2 fold increase; p=0.537).

This shows that KB-V13A12 efficiently increases endogenous plasma levels of VWF and FVIII following a single subcutaneous injection for a period of at least 10 days in this mouse model for von Willebrand disease type 1.

### Example 9 Correction of Hemostasis after Injection with KB-V13A12

To test if the increased levels of VWF and FVIII after injection with KB-V13A12 are functionally active, a tail clip bleeding model was applied. Three groups of mice (both male and females, 8-12 weeks old) were included in this experiment. Wild-type 129Sv mice were used as control mice to establish bleeding in wild-type mice (referred to WT; n=13), untreated transgenic 129Sv mice expressing human von Willebrand factor (VWF) and human Glycoprotein Ibalpha to establish bleeding tendency in these mice (referred to as VWD-type 1; n=26), and transgenic 129Sv mice expressing human von Willebrand factor (VWF) and human Glycoprotein Ibalpha that received 100 μg KB-V13A12 via subcutaneous injection (referred to VWD-type 1+KB-V13A12; n=9).

Three days after injection of KB-V13A12, the terminal 3 mm of the tail tip of ketamine/xylazine-anesthetized mice in each of these three groups was amputated. The amputated tail was immersed immediately after transection in a 50-ml tube full of warm physiological saline. Blood was collected for 30 min at 37° C. After 30 min, the mixture of blood and physiological saline was centrifuged at 1,500 g. The red blood cells pellet was then lysed in H2O and the amount of hemoglobin was obtained by reading the absorbance at 416 nm. The volume of blood lost in each sample was calculated from a standard curve, which is obtained by lysing defined volumes (20 microliters, 40 microliters, 60 microliters, 80 microliters and 100 microliters) of mouse blood in H2O to extract hemoglobin as described above. Blood loss for each individual mouse is shown in FIG. 6.

Compared to WT-mice, blood loss was significantly increased in VWD-type 1 mice (102±123 microliter versus 19±37 microliter for VWD-type 1 and WT mice, respectively; p=0.0251). In contrast, blood loss in VWD-type 1±KB-V13A12 mice was similar to that of WT mice (47±49 microliter; p=0.711). Thus, KB-V13A12 treatment results in improved hemostasis in VWD-type 1 mice.

### Example 10 In Vivo Survival of Recombinant Von Willebrand Factor in the Absence Or Presence of KB-V13A12

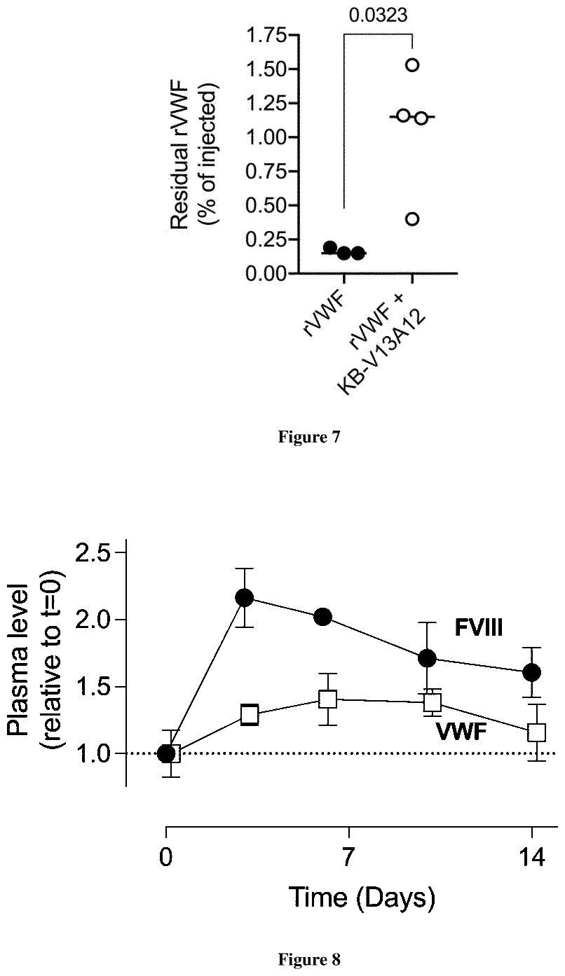

Purified recombinant von Willebrand factor (VWF; 0.1 mg/ml) was incubated in the absence or presence of KB-V13A12 (0.12 mg/ml, a tenfold molar excess) for 30 min at room temperature in PBS. VWF-deficient mice were then anesthetized with isoflurane and the VWF-containing solutions were infused intravenously via the retro-orbital sinus at a dose of 0.5 mg VWF/kg bodyweight. At different time-points (3 min and 24h), blood samples were obtained via retro-orbital puncture from isoflurane-anesthetized mice and plasma was prepared by centrifugation (1,500 g for 20 min at room temperature). Three mice received VWF alone, whereas 4 mice received VWF in the presence of KB-V13A12. Residual plasma concentrations of VWF were determined employing an in-house ELISA that measures VWF using polyclonal rabbit anti-VWF antibodies (Dakocytomation, Glostrup, Denmark) as catching agent, and peroxidase-labeled polyclonal rabbit anti-VWF antibodies (Dakocytomation, Glostrup, Denmark) as probing agent. Recoveries at 3 min after injection were similar for VWF alone and the VWF/KB-V13A12 combination (67±42% and 86±23% of the amount injected, respectively). In contrast, levels of VWF were statistically significant 6.6-fold lower in mice that received VWF alone (0.16±0.02% of the amount injected) compared to the VWF-deficient mice that received VWF in the presence of KB-V13A12 (1.06±0.5% of the amount injected; p=0.0323 as analysed in a unpaired t-test with Welch's correction; FIG. 7). This shows that associating VWF with KB-V13A12 prolongs the survival of VWF in the circulation.

### Example 11 KB-V80A12 Differentially Increases VWF and FVIII Plasma Levels

A construct encoding KB-VWF-080 fused to OptiAlb-12 (SEQID NO: 47) was cloned into the pHEN6-plasmid so to comprise a C-terminal histidine and cMyc-tag. The bispecific single-domain antibody was expressed and purified as described in Example 1. The purified protein was at a concentration of 5.2 milligram/ml, and is designated as KB-V80A12.

Control immunosorbent assays demonstrated that KB-V80A12 can simultaneously bind to both albumin and VWF.

The purified protein was then used for in vivo studies. Transgenic 129Sv mice expressing human von Willebrand factor (VWF) and human Glycoprotein Ibalpha were used for this study. VWF levels in these mice are 15±4% compared to normal human plasma and factor VIII (FVIII) activity levels are 44±8% compared to normal human plasma. This mouse model thus represents a model for von Willebrand disease type 1.

These mice received 100 microgram of KB-V80A12 via subcutaneous injection. Blood samples were taken 24h before injection, and plasma levels of VWF and FVIII from this samples were defined as t=0. Additional blood samples were taken at day 3, day 6, day 10 and day 14. Plasma samples were prepared and analysed for levels of VWF antigen and FVIII activity.

Relative changes in plasma levels of VWF and FVIII are depicted in FIG. 8, respectively. Depicted is the relative increase of VWF antigen or FVII activity relative to t=0 versus day after injection. Plasma levels of VWF increased 1.3±0.1 fold at day 3 (not significant compared to T=0), 1.4±0.2 fold at day 6 (p=0.04 compared to T=0), 1.4±0.1 fold at day 10 (p=0.05 compared to T=0) and 1.2±0.2 fold at day 14 (not significant compared to T=0). Plasma levels of FVIII increased 2.2±0.2 fold at day 3 (p<0.0001 compared to T=0), 2.0±0.1 fold at day 6 (p=0.0001 compared to T=0), 1.7±0.3 fold at day 10 (p=0.0022 compared to T=0) and 1.6±0.2 fold at day 14 (p=0.0065 compared to T=0).

This shows that KB-V80A12 efficiently increases endogenous plasma levels of FVIII, accompanied by a minor increase of VWF levels following a single subcutaneous injection for a period of at least 10 days in this mouse model for von Willebrand disease type 1.

### Example 12 Epitope OptiAlb-12

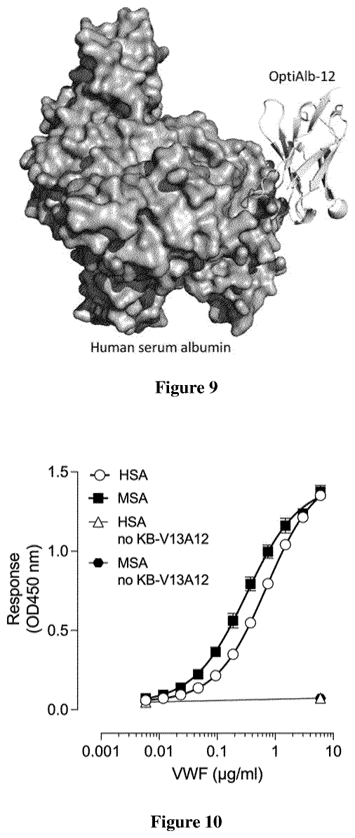

Three-dimensional structure of OptiAlb-12 was modeled by using the VH-structure available in crystal structure7CJ2 (https://www.rcsb.org/structure/7CJ2) and the albumin structure was obtained from crystal structure 1A06 (https://www.rcsb.org/structure/lao6). The OptiAlb-12 structure was docked on the albumin structure using MAbTope (Bourquard et al. J Immunol 2018 201:3096-3105) in order to identify the epitope for OptiAlb-12 on albumin. The 30 top-ranked structures reveal that they all clustered at the same epitope within the DIII epitope of albumin, involving albumin residues: Thr491-Lys499, Glu516-Phe526 and His559-Lys565. The complex between human albumin and OptiAlb-12 is visualized in FIG. 9. Residues were numbered according to the Uniprot sequence (P02768).

### Example 13: Simultaneous Binding of KB-V13A12 to VWF and Albumin

Microtiter wells were coated with human serum albumin (HSA) or murine serum albumin (MSA) both at 6 microgram/ml overnight at 4° C. Wells were emptied and incubated for 1 h at 37° C. with PBS/1% bovine serum albumin. After washing four times with PBS/0.1% Tween-20, wells were incubated with KB-V13A12 (200 nM) for 1 h at 37° C. Control wells were incubated with PBS/0.1% Tween-20 for the same period of time. After washing four times with PBS/0.1% Tween-20, wells were incubated with serial dilutions of purified VWF (0-6 microgram/ml) for 1 h at 37° C. After washing four times with PBS/0.1% Tween-20, bound VWF was probed with rabbit polyclonal horseradish-peroxidase labelled anti-VWF antibodies (DAKO, ref P0226) for 1 h at 37° C. After washing six times with PBS/0.1% Tween-20, wells were incubated with 3,3′,5,5′-tetramethylbenzidine for 5 minutes under gentle shaking. Hydrolysis was stopped by the addition of 1 M H₂SO₄ and absorbance at 450 nm was measured (OD450).

Analysis of the data revealed a dose-dependent binding of VWF in the presence but not absence of KB-V13A12 (FIG. 10). Binding was also absent in uncoated wells. Together these data show that KB-V13A12 simultaneously binds HSA and VWF as well as MSA and VWF.

### Example 14: Binding of Humanized KB-V13A12 Variants to VWF

Microtiter wells were coated with human VWF (6 microgram/ml) overnight at 4° C. Wells were emptied and incubated for 1 h at 37° C. with PBS/1% bovine serum albumin. After washing four times with PBS/0.1% Tween-20, wells were incubated with various concentrations of KB-V13A12 (SEQ ID: 30), KB-V13A12/T78S (SEQ ID: 33), KB-V13A12/E46V-T78S (SEQ ID: 36), KB-V13A12/V5L-E46V (SEQ ID: 44), KB-V13A12/V5L-T78S (SEQ ID: 45), KB-V13A12/V5L-E46V-T78S (SEQ ID: 46) (0-100 nM) in PBS/0.5% BSA/0.1% Tween-20 After washing four times with PBS/0.1% Tween-20, bound nanobodies were probed using polyclonal peroxidase-labelled anti-cMyc antibodies and detected via hydrolysis of 3,3′,5,5′-tetramethylbenzidine for 5 minutes under gentle shaking. Hydrolysis was stopped by the addition of 1M H₂SO₄ and absorbance at 450 nm was measured (OD450).

Data analysis revealed that all humanized variants were similar to KB-V13A12 in binding to VWF (FIG. 11).

### Example 15: Binding of KB-V13A12 to Albumin and VWF at Neutral and Low pH

Microtiter wells were coated with human serum albumin (HSA) at 6 microgram/ml overnight at 4° C. Wells were emptied and incubated for 1 h at 37° C. with PBS/1% bovine serum albumin. After washing four times with PBS/0.1% Tween-20, wells were incubated with KB-V13A12 (100 nM) for 1 h at 37° C. Control wells were incubated with PBS/0.1% Tween-20 for the same period of time. After washing four times with PBS/0.1% Tween-20, wells were incubated with serial dilutions of purified VWF 0-6 microgram/ml) for 1 h at 37° C. After washing four times with PBS/0.1% Tween-20, wells were incubated in 42 mM citric acid/58 mM Na₂HPO₄/150 mM NaCl (pH 5.6) or 9.2 mM citric acid/90.9 mM Na₂HPO₄/150 mM NaCl (pH 7.4) for 20 min at 37° C. This step was repeated three times for 20 min. Bound VWF was then probed with rabbit polyclonal horseradish-peroxidase labelled anti-VWF antibodies (DAKO, ref P0226) for 1 h at 37° C. After washing six times with PBS/0.1% Tween-20, wells were incubated with 3,3′,5,5′-tetramethylbenzidine for 5 minutes under gentle shaking. Hydrolysis was stopped by the addition of 1 M H₂SO₄ and absorbance at 450 nm was measured (OD450).

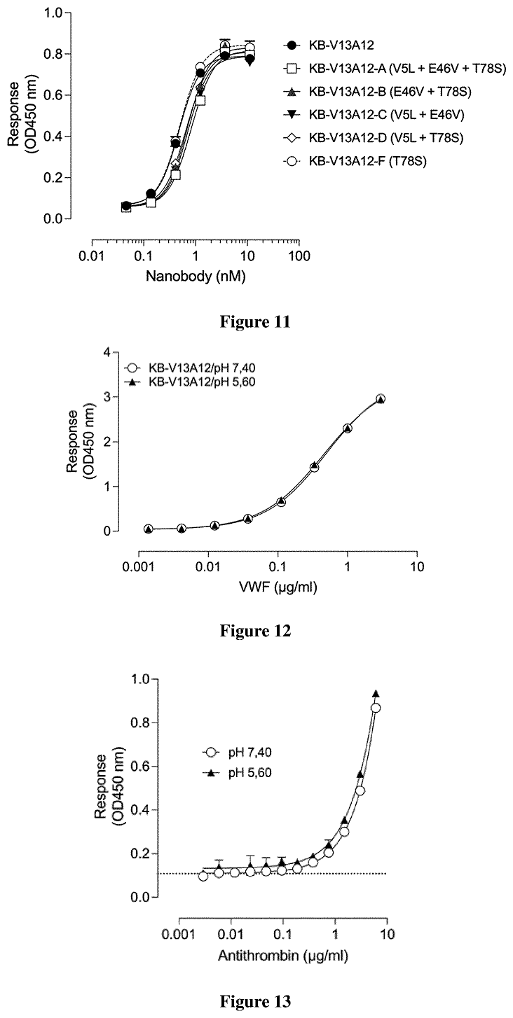

Data analysis revealed that binding to albumin and VWF was similar at pH 7.4 and 5.6 (FIG. 12).

### Example 16 Bridging Human Albumin and Antithrombin Using KB-AT01A12

A construct encoding KB-AT-01 fused to OptiAlb-12 (SEQ ID NO:57) was cloned into the pHEN6-plasmid so to comprise a C-terminal histidine and cMyc-tag. The bispecific single-domain antibody was expressed and purified as described in Example 1. The purified protein was at a concentration of 1 mg/ml, and is designated as KB-AT01A12.

Microtiter wells were coated with human serum albumin (HSA) at 6 microgram/ml overnight at 4° C. Wells were emptied and incubated for 1 h at 37° C. with PBS/1% bovine serum albumin. After washing four times with PBS/0.1% Tween-20, wells were incubated with KB-AT01A12 (100 nM) for 1 h at 37° C. Control wells were incubated with PBS/0.1% Tween-20 for the same period of time. After washing four times with PBS/0.1% Tween-20, wells were incubated with serial dilutions of purified antithrombin (0-6 microgram/ml) for 1 h at 37° C. After washing four times with PBS/0.1% Tween-20, wells were incubated in 42 mM citric acid/58 mM Na₂HPO₄/150 mM NaCl (pH 5.6) or 9.2 mM citric acid/90.9 mM Na₂HPO₄/150 mM NaCl (pH 7.4) for 20 min at 37° C. This step was repeated three times for 20 min. Bound antithrombin was then probed with rabbit polyclonal horseradish-peroxidase labelled anti-antithrombin antibodies (US Biologicals) for 1 h at 37° C. After washing six times with PBS/0.1% Tween-20, wells were incubated with 3,3′,5,5′-tetramethylbenzidine for 5 minutes under gentle shaking. Hydrolysis was stopped by the addition of 1 M H₂SO₄ and absorbance at 450 nm was measured (OD450). Data analysis revealed that binding to albumin and antithrombin was similar at pH 7.4 and 5.6 (FIG. 13).

### Example 17

A construct encoding KB-FX-E3 fused to OptiAlb-12 (SEQID NO: 62) was cloned into the pHEN6-plasmid so to comprise a C-terminal histidine and cMyc-tag. The bispecific single-domain antibody was expressed and purified as described in Example 1. The purified protein was at a concentration of 0.6 mg/ml, and is designated as KB-FXE3A12.

The purified protein was then used to test its capacity to bridge antithrombin to albumin.

Microtiter wells were coated with human serum albumin (HSA) at 6 microgram/ml overnight at 4° C. Wells were emptied and incubated for 1 h at 37° C. with PBS/0.1% Tween-20/1% bovine serum albumin. After washing four times with PBS/0.1% Tween-20, wells were incubated with KB-FXE3A12 (100 nM) for 1 h at 37° C. in PBS/0.05% Tween-20/0.5% BSA. After washing four times with PBS/0.1% Tween-20, wells were incubated with serial dilutions of purified factor X (0-6 microgram/ml) for 1 h at 37° C. in PBS/0.05% Tween-20/0.5% BSA. After washing four times with PBS/0.1% Tween-20, bound factor X was probed with rabbit polyclonal horseradish-peroxidase labelled anti-factor X antibodies for 1 h at 37° C. in PBS/0.05% Tween-20/0.5% BSA. After washing six times with PBS/0.1% Tween-20, wells were incubated with 3,3′,5,5′-tetramethylbenzidine for 5 minutes under gentle shaking. Hydrolysis was stopped by the addition of 1 M H₂SO₄ and absorbance at 450 nm was measured (OD450).

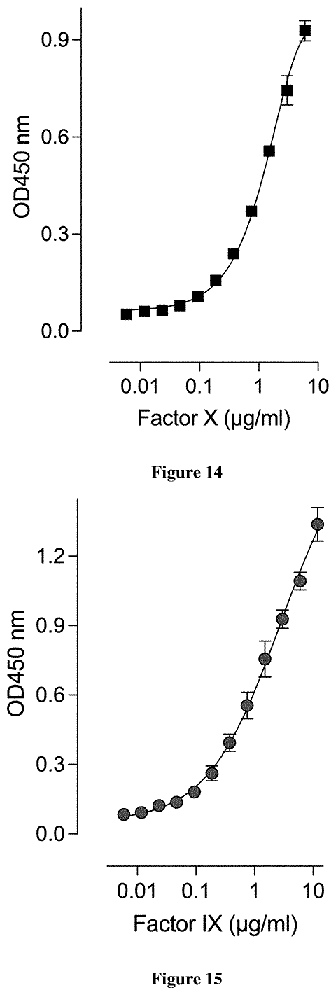

Analysis of the data revealed a dose-dependent binding of factor X in the presence KB-FXE3A12 (FIG. 14). Binding was also absent in uncoated wells. Together these data show that KB-FXE3A12 simultaneously binds HSA and factor X.

### Example 18

A construct encoding KB-F9-D9 fused to OptiAlb-12 (SEQID NO: 67) was cloned into the pHEN6-plasmid so to comprise a C-terminal histidine and cMyc-tag. The bispecific single-domain antibody was expressed and purified as described in Example 1. The purified protein was at a concentration of 2.7 mg/ml, and is designated as KB-F9D9A12.

Microtiter wells were coated with human serum albumin (HSA) at 6 microgram/ml overnight at 4° C. Wells were emptied and incubated for 1 h at 37° C. with PBS/1% bovine serum albumin. After washing four times with 20 mM Hepes/0.15 M NaCl/2.5 mM CaCl₂/1% bovine serum albumin, wells were incubated with KB-F9D9A12 (100 nM) for 1 h at 37° C. 20 mM Hepes/0.15 M NaCl/2.5 mM CaCl₂/0.5% BSA/0.05% Tween-20. After washing four times with 20 mM Hepes/0.15 M NaCl/2.5 mM CaCl₂/0.1% Tween-20, wells were incubated with serial dilutions of purified factor IX (0-12 microgram/ml) for 1 h at 37° C. in 20 mM Hepes/0.15 M NaCl/2.5 mM CaCl₂/0.5% BSA/0.05% Tween-20. After washing four times with 20 mM Hepes/0.15 M NaCl/2.5 mM CaCl₂/0.1% Tween-20, bound factor IX was probed with rabbit polyclonal horseradish-peroxidase labelled anti-factor IX antibodies for 1 h at 37° C. in in 20 mM Hepes/0.15 M NaCl/2.5 mM CaCl₂/0.5% BSA/0.05% Tween-20. After washing six times with 20 mM Hepes/0.15 M NaCl/2.5 mM CaCl₂/0.1% Tween-20, wells were incubated with 3,3′,5,5′-tetramethylbenzidine for 5 minutes under gentle shaking. Hydrolysis was stopped by the addition of 1 M H₂SO₄ and absorbance at 450 nm was measured (OD450).

Analysis of the data revealed a dose-dependent binding of factor IX in the presence KB-F9D9A12 (FIG. 15). Binding was also absent in uncoated wells. Together these data show that KB-F9D9A12 simultaneously binds HSA and factor IX.

### Example 19: Affinity of OptiAlb-12 to Human and Murine Albumin

Real-time binding studies were performed using openSPR-equipement. OptiAlb-12 (SEQ ID NO: 21) was immobilized using EDC/NHS-amino coupling (1500-2000 RU), and non-occupied sites were blocked via subsequent incubations with a non-relevant single-domain antibody and ethanolamine (1M). A control channel was blocked using the non-relevant single-domain antibody and ethanolamine, without the presence of OptiAlb-12. Binding of albumin to the OptiAlb-12 channel was corrected for binding to the control channel (<3% of binding to OptiAlb-12-coated channels). SPR-analysis was performed in PBS/0.1% Tween-20. Various concentrations of HSA or MSA (0-1.2 nM-3.7 nM-11.1 nM-33.3 nM-100 nM) were perfused over both control and OptiAlb-12-coated channels at 25° C. with a flow rate of 40 microliter/min. Association was 2 min and dissociation was 10 min. Channels were regenerated using 10 mM Glycine-HCl, pH 1.5 (10 sec at 200 microliter/min).

Sensorgrams of two independent experiments (Experiment 1 and Experiment 2) were analysed using TraceDrawer-software to calculate affinity constants, which are summarized in Table 4.

TABLE 4
Affinity constants (K_{D}) for the interactions
between OptiAlb-12 and HSA or MSA

| | HSA | MSA |
|---|---|---|
| Experiment 1 | K_{D} < 1 nM | K_{D} < 1 nM |
| Experiment 2 | K_{D} < 1 nM | K_{D} < 1 nM |

### REFERENCES

Throughout this application, various references describe the state of the art to which this invention pertains. The disclosures of these references are hereby incorporated by reference into the present disclosure.

1. An isolated single-domain antibody (sdAb) directed against albumin comprises:
a CDR1 having a sequence set forth as SEQ ID NO: 2, a CDR2 having a sequence set forth as SEQ ID NO: 3 and a CDR3 having a sequence set forth as SEQ ID NO: 4;
a CDR1 having a sequence set forth as SEQ ID NO:6, a CDR2 having a sequence set forth as SEQ ID NO: 7 and a CDR3 having a sequence set forth as SEQ ID NO: 8;
a CDR1 having a sequence set forth as SEQ ID NO:10, a CDR2 having a sequence set forth as SEQ ID NO: 11 and a CDR3 having a sequence set forth as SEQ ID NO: 12;
a CDR1 having a sequence set forth as SEQ ID NO:14, a CDR2 having a sequence set forth as SEQ ID NO: 15 and a CDR3 having a sequence set forth as SEQ ID NO: 16; or
a CDR1 having a sequence set forth as SEQ ID NO:18, a CDR2 having a sequence set forth as SEQ ID NO: 19 and a CDR3 having a sequence set forth as SEQ ID NO: 20.

2. The isolated single-domain antibody directed against albumin according to claim 1, wherein said single-domain antibody comprises:
a CDR1 having at least 70% of identity with sequence set forth as SEQ ID NO: 2, a CDR2 having at least 70% of identity with sequence set forth as SEQ ID NO: 3 and a CDR3 having at least 70% of identity with sequence set forth as SEQ ID NO: 4;
a CDR1 having at least 70% of identity with sequence set forth as SEQ ID NO:6, a CDR2 having at least 70% of identity with sequence set forth as SEQ ID NO: 7 and a CDR3 having at least 70% of identity with sequence set forth as SEQ ID NO: 8;
a CDR1 having at least 70% of identity with sequence set forth as SEQ ID NO:10, a CDR2 having at least 70% of identity with sequence set forth as SEQ ID NO: 11 and a CDR3 having at least 70% of identity with sequence set forth as SEQ ID NO: 12;
a CDR1 having at least 70% of identity with sequence set forth as SEQ ID NO:14, a CDR2 having at least 70% of identity with sequence set forth as SEQ ID NO: 15 and a CDR3 having at least 70% of identity with sequence set forth as SEQ ID NO: 16; or
a CDR1 having at least 70% of identity with sequence set forth as SEQ ID NO:18, a CDR2 having at least 70% of identity with sequence set forth as SEQ ID NO: 19 and a CDR3 having at least 70% of identity with sequence set forth as SEQ ID NO: 20.

3. The isolated single-domain antibody directed against albumin according to claim 1 wherein said single-domain antibody is:
OptiAlb-03 (SEQ ID NO: 5),
OptiAlb-07 (SEQ ID NO:9);
OptiAlb-09 (SEQ ID NO:13);
OptiAlb-11 (SEQ ID NO:17) or
OptiAlb-12 (SEQ ID NO:21).

4. A chimeric polypeptide comprising a polypeptide and at least one single-domain antibody directed against albumin.

5. The chimeric polypeptide according to claim 4 wherein at least one single-domain antibody directed against albumin is a single domain antibody according to claims 1 to 3.

6. The chimeric polypeptide according to claim 4 wherein the polypeptide is a clotting factor selected from the group consisting of but limited to: VWF, FVII, FVIII, antithrombin, fibrinogen, protein C, protein S, complement proteins (in particular C2, C9, Mannose-binding Lectin (MBL), C1-inhibitor, factor H-related protein-3), Serpins (in particular SerpinA1, SerpinA3, SerpinA5, SerpinA6, SerpinA7, SerpinA8, SerpinA10, SerpinC1 (=antithrombin), SerpinD1, SerpinE1, SerpinF2, SerpinG1, SerpinI1), fibrinolysis-related proteins (tissue-type plasminogen activator, urokinase-type plasminogen activator, plasminogen), protein-hormones, growth-factors, interleukins, insulin, glucagon, osteoprotegerin (OPG), Angiopoietin-2 (ANGPT2) or furin.

7. The chimeric polypeptide according to claim 6, comprises at least one single-domain antibody according to claims 1 to 3, wherein said single-domain antibody is fused at the N terminal end, at the C terminal end, both at the N terminal end and at the C terminal end of the therapeutic polypeptide or is inserted within the sequence of the therapeutic polypeptide.

8. The chimeric polypeptide according to claims 4 to 7, wherein the chimeric polypeptide comprising two, three, four, or five single-domain antibody directed against albumin.

9. The chimeric polypeptide according to claims 4 to 8, wherein the polypeptide comprises at least one single-domain antibody directed against a first antigen and at least one further binding site directed against a second antigen.

10. The chimeric polypeptide according to claims 4 to 9 is a bispecific polypeptide.

11. The chimeric polypeptide according to claim 10, wherein the bispecific polypeptide has the following sequence consisting of but not limited to: SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40, SEQ ID NO: 43, SEQ ID NO: 44; SEQ ID NO: 45, SEQ ID NO: 46, SEQ ID NO:47, SEQ ID NO:52, SEQ ID NO:57, SEQ ID NO:62, SEQ ID NO:67, SEQ ID NO:68, SEQ ID NO:69, and SEQ ID NO:70.

12. A nucleic acid molecule encoding the single domain antibody according to claims 1 to 3 and/or the chimeric polypeptide according to claims 4 to 11.

13. A vector comprises the nucleic acid according to claim 12.

14. An isolated single-domain antibody (sdAb) directed against albumin according to claims 1 to 3 or a chimeric polypeptide according to claims 4 to 11 for use to increase the circulatory half-life of an endogenous plasma protein, an exogenous polypeptide, a single-domain antibody that targets another polypeptide or a polypeptide.

15. An isolated single-domain antibody (sdAb) directed against albumin according to claims 1 to 3 or a chimeric polypeptide according to claims 4 to 11 for use as drug.

16. An isolated single-domain antibody (sdAb) directed against albumin according to claims 1 to 3 or a chimeric polypeptide according to claims 4 to 11 for use in a method for preventing or treating bleeding disorders.

17. A method of preventing or treating bleeding disorders in a subject in need thereof, comprising administering to said subject a therapeutically effective amount of an isolated single-domain antibody (sdAb) directed against albumin according to claims 1 to 3 or a chimeric polypeptide according to claims 4 to 11.

18. The method according to claim 17 wherein the bleeding disorder is the bleeding disorder is selected from the group consisting of but not limited to: von Willebrand disease, hemophilia A, or hemophilia B, protein C deficiency, protein S deficiency, antithrombin deficiency, factor XI deficiency, C1-esterase inhibitor deficiency, insulin-deficiency, alpha-1-antitrypsin deficiency, complement C2 deficiency or sickle cell disease.

19. The isolated single-domain antibody directed against albumin according to claims 1 to 3 or the chimeric polypeptide according to claims 4 to 11 for use in a method for preventing or treating a subject having low level of a therapeutic protein.

20. The isolated single-domain antibody directed against albumin according to claims 1 to 3 or the chimeric polypeptide according to claims 4 to 11 for use according to claim 19 wherein the therapeutic protein is a clotting factor.

21. The isolated single-domain antibody directed against albumin according to claims 1 to 3 or the chimeric polypeptide according to claims 4 to 11 for use according to claim 20 wherein the clotting factor is selected from the group consisting of but limited to: VWF, FVII, FVIII, antithrombin, fibrinogen, protein C, protein S, complement proteins (C2, C9, Mannose-binding Lectin (MBL), C1-inhibitor, factor H-related protein-3), Serpins (SerpinA1, SerpinA3, SerpinA5, SerpinA6, SerpinA7, SerpinA8, SerpinA10, SerpinC1 (=antithrombin), SerpinD1, SerpinE1, SerpinF2, SerpinG1, SerpinI1), fibrinolysis-related proteins (tissue-type plasminogen activator, urokinase-type plasminogen activator, plasminogen), protein-hormones or growth-factors and interleukins.

22. A pharmaceutical composition comprising a single-domain antibody directed against albumin according to claims 1 to 3 or a chimeric polypeptide according to claims 4 to 11, and a pharmaceutically acceptable carrier.

23. The pharmaceutical composition according to claim 22 for use in the prevention or treatment of bleeding disorders.


SEQUENCE LISTING (71 sequences)

SEQ ID NO: 1 - protein, 115 aa, synthetic construct [sequence omitted]

SEQ ID NO: 2 - protein, 8 aa, synthetic construct
GFTFSSFG

SEQ ID NO: 3 - protein, 8 aa, synthetic construct
ISGSGSDT

SEQ ID NO: 4 - protein, 11 aa, synthetic construct
TIGGSLSPSSQ

SEQ ID NO: 5 - protein, 115 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 6 - protein, 8 aa, synthetic construct
GFTFSSFG

SEQ ID NO: 7 - protein, 8 aa, synthetic construct
ISGSGDDT

SEQ ID NO: 8 - protein, 11 aa, synthetic construct
TIGGSLSPSSQ

SEQ ID NO: 9 - protein, 115 aa, synthetic construct
EVQLVESGGGLVQPGNSLRLSCAASGFTFSSFGMSWVRQAPGKGLEWVSSISGSGDDTLYADSVKGRFTISRDNAKTTLYLQMNSLRPEDTAVYYCTIGGSLSPSSQGTLVTVSS

SEQ ID NO: 10 - protein, 8 aa, synthetic construct
GFTFSSFG

SEQ ID NO: 11 - protein, 8 aa, synthetic construct
ISGSGSDT

SEQ ID NO: 12 - protein, 11 aa, synthetic construct
TIGGSKSRSSG

SEQ ID NO: 13 - protein, 115 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSKSRSSGGTLVTVSS

SEQ ID NO: 14 - protein, 8 aa, synthetic construct
GFTFSSFG

SEQ ID NO: 15 - protein, 8 aa, synthetic construct
ISGSGHDT

SEQ ID NO: 16 - protein, 11 aa, synthetic construct
TIGGSLTRSSQ

SEQ ID NO: 17 - protein, 115 aa, synthetic construct
EVQLVESGGGLVQPGNSLRLSCAASGFTFSSFGMSWVRQAPGKGPEWVSSISGSGHDTLYADSVKGRFTISRDNAKTTLYLQMNSLRPEDTAVYYCTIGGSLTRSSQGTLVTVSS

SEQ ID NO: 18 - protein, 8 aa, synthetic construct
GFTFSRFG

SEQ ID NO: 19 - protein, 8 aa, synthetic construct
ISGSGSDT

SEQ ID NO: 20 - protein, 11 aa, synthetic construct
TIGGSLSPSSQ

SEQ ID NO: 21 - protein, 115 aa, synthetic construct
EVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 22 - protein, 10 aa, synthetic construct
GRTFIRYAMA

SEQ ID NO: 23 - protein, 16 aa, synthetic construct
IPQSGGRSYYADSVKG

SEQ ID NO: 24 - protein, 19 aa, synthetic construct
TSTYYGRSAYSSHSGGYDY

SEQ ID NO: 25 - protein, 128 aa, synthetic construct
QVQLVQSGGGLVQAGDSLRLSCAASGRTFIRYAMAWFRQAPGKEREFVAAIPQSGGRSYYADSVKGRFTISRDNAKNTVYLQMNSLKPEDTAVYSCAATSTYYGRSAYSSHSGGYDYWGQGTQVTVSS

SEQ ID NO: 26 - protein, 8 aa, synthetic construct
GRTFSDYA

SEQ ID NO: 27 - protein, 8 aa, synthetic construct
ISRSGGRL

SEQ ID NO: 28 - protein, 18 aa, synthetic construct
AARTNWNPPRPLPEEYNY

SEQ ID NO: 29 - protein, 125 aa, synthetic construct
QVQLVQSGGGLVQAGGSLKLSCAASGRTFSDYAMGWFRQAPGKERDFVASISRSGGRLSYADSVKGRFTISRDNAKNTVYLQMNSLKPEDTAVYYCAARTNWNPPRPLPEEYNYWGQGTQVTVSS

SEQ ID NO: 30 - protein, 262 aa, synthetic construct
QVQLVQSGGGLVQAGDSLRLSCAASGRTFIRYAMAWFRQAPGKEREFVAAIPQSGGRSYYADSVKGRFTISRDNAKNTVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 31 - protein, 262 aa, synthetic construct
QVQLVQSGGGLLQAGDSLRLSCAASGRTFIRYAMAWFRQAPGKEREFVAAIPQSGGRSYYADSVKGRFTISRDNAKNTVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 32 - protein, 262 aa, synthetic construct
QVQLVQSGGGLVQAGDSLRLSCAASGRTFIRYAMAWFRQAPGKERVFVAAIPQSGGRSYYADSVKGRFTISRDNAKNTVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 33 - protein, 262 aa, synthetic construct
QVQLVQSGGGLVQAGDSLRLSCAASGRTFIRYAMAWFRQAPGKEREFVAAIPQSGGRSYYADSVKGRFTISRDNAKNSVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 34 - protein, 262 aa, synthetic construct
QVQLVQSGGGLLQAGDSLRLSCAASGRTFIRYAMAWFRQAPGKERVFVAAIPQSGGRSYYADSVKGRFTISRDNAKNTVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 35 - protein, 262 aa, synthetic construct
QVQLVQSGGGLLQAGDSLRLSCAASGRTFIRYAMAWFRQAPGKEREFVAAIPQSGGRSYYADSVKGRFTISRDNAKNSVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 36 - protein, 262 aa, synthetic construct
QVQLVQSGGGLVQAGDSLRLSCAASGRTFIRYAMAWFRQAPGKERVFVAAIPQSGGRSYYADSVKGRFTISRDNAKNSVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 37 - protein, 262 aa, synthetic construct
QVQLVQSGGGLLQAGDSLRLSCAASGRTFIRYAMAWFRQAPGKERVFVAAIPQSGGRSYYADSVKGRFTISRDNAKNSVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 38 - protein, 255 aa, synthetic construct
QVQLQESGGGLVQAGGSLRLSCAASGRTFSSYAMGWVRQAPGKEREFVAAISYNGGRTNYADSVKGRFTISRDNAKNTGYLQMNSLKPEDTAVYYCAANPRMWGSVDFRSWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 39 - protein, 392 aa, synthetic construct
QVQLQESGGGLVQAGGSLRLSCAASGRTFSSYAMGWVRQAPGKEREFVAAISYNGGRTNYADSVKGRFTISRDNAKNTGYLQMNSLKPEDTAVYYCAANPRMWGSVDFRSWGQGTQVTVSSGGGSGGGSGGGSGGGSQVQLQESGGGLVQAGGSLRLSCAASGRTFSSYAMGWVRQAPGKEREFVAAISYNGGRTNYADSVKGRFTISRDNAKNTGYLQMNSLKPEDTAVYYCAANPRMWGSVDFRSWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 40 - protein, 403 aa, synthetic construct
EVQLQASGGGFVQPGGSLRLSCAASGSTWFREIMGWFRQAPGKEREFVSAISSDPTWHAYYADSVKGRFTISRDNSKNTVYLQMNSLRAEDTATYYCAPLAGTESIHWWDPWHESSYWGQGTQVTVSSGGGSGGGSGGGSGGGSEVQLQASGGGFVQPGGSLRLSCAASGDTWSLEIMGWFRQAPGKEREFVSAISSEDGWHAYYADSVKGRFTISRDNSKNTVYLQMNSLRAEDTATYYCAKIENWIQAVEGEMSDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 41 - protein, 12 aa, synthetic construct
EVQLVESGGGLV

SEQ ID NO: 42 - protein, 12 aa, synthetic construct
QVQLQQSGGGFV

SEQ ID NO: 43 - protein, 262 aa, synthetic construct
QVQLLQSGGGLVQAGDSLRLSCAASGRTFIRYAMAWFRQAPGKEREFVAAIPQSGGRSYYADSVKGRFTISRDNAKNTVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 44 - protein, 262 aa, synthetic construct
QVQLLQSGGGLVQAGDSLRLSCAASGRTFIRYAMAWFRQAPGKERVFVAAIPQSGGRSYYADSVKGRFTISRDNAKNTVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 45 - protein, 262 aa, synthetic construct
QVQLLQSGGGLVQAGDSLRLSCAASGRTFIRYAMAWFRQAPGKEREFVAAIPQSGGRSYYADSVKGRFTISRDNAKNSVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 46 - protein, 262 aa, synthetic construct
QVQLLQSGGGLVQAGDSLRLSCAASGRTFIRYAMAWFRQAPGKERVFVAAIPQSGGRSYYADSVKGRFTISRDNAKNSVYLQMNSLKPEDTAVYSCAATSTYYGRSAYSSHSGGYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 47 - protein, 259 aa, synthetic construct
QVQLVQSGGGLVQAGGSLKLSCAASGRTFSDYAMGWFRQAPGKERDFVASISRSGGRLSYADSVKGRFTISRDNAKNTVYLQMNSLKPEDTAVYYCAARTNWNPPRPLPEEYNYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 48 - protein, 8 aa, synthetic construct
GRTFSSNA

SEQ ID NO: 49 - protein, 8 aa, synthetic construct
AAISWMST

SEQ ID NO: 50 - protein, 15 aa, synthetic construct
ARREDRRVLTTDYDY

SEQ ID NO: 51 - protein, 121 aa, synthetic construct
QVQLVQSGGGLVQAGGSLRLSCAASGRTFSSNAMAWFRQAPGKEREFVAAISWMSTTYADSVAGRFTISRDNAKNTVYLQMNSLKPEDTAVYYCAARREDRRVLTTDYDYWGQGTQVTVSS

SEQ ID NO: 52 - protein, 255 aa, synthetic construct
QVQLVQSGGGLVQAGGSLRLSCAASGRTFSSNAMAWFRQAPGKEREFVAAISWMSTTYADSVAGRFTISRDNAKNTVYLQMNSLKPEDTAVYYCAARREDRRVLTTDYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 53 - protein, 8 aa, synthetic construct
GRTFRNYV

SEQ ID NO: 54 - protein, 8 aa, synthetic construct
INRSGAIT

SEQ ID NO: 55 - protein, 16 aa, synthetic construct
AAGETTWSIRRDDYDY

SEQ ID NO: 56 - protein, 123 aa, unidentified
QVQLVQSGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSS

SEQ ID NO: 57 - protein, 257 aa, synthetic construct
QVQLVQSGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 58 - protein, 8 aa, synthetic construct
GSISRGDL

SEQ ID NO: 59 - protein, 7 aa, synthetic construct
ITPGANT

SEQ ID NO: 60 - protein, 16 aa, synthetic construct
AAASGKGPGRGRKHKY

SEQ ID NO: 61 - protein, 122 aa, synthetic construct
QVQLQESGGGLVQAGGSLRLSCAASGSISRGDLMAWFRQAPGKERELVATITPGANTYYADSVKGRFTISRDNTKNTMYLQMNSLKPEDTAVYFCAAASGKGPGRGRKHKYWGQGTQVTVSS

SEQ ID NO: 62 - protein, 256 aa, synthetic construct
QVQLQESGGGLVQAGGSLRLSCAASGSISRGDLMAWFRQAPGKERELVATITPGANTYYADSVKGRFTISRDNTKNTMYLQMNSLKPEDTAVYFCAAASGKGPGRGRKHKYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 63 - protein, 8 aa, synthetic construct
GLIFSFNA

SEQ ID NO: 64 - protein, 7 aa, synthetic construct
ITSGGST

SEQ ID NO: 65 - protein, 9 aa, synthetic construct
RSSQSGVEY

SEQ ID NO: 66 - protein, 115 aa, synthetic construct
QVQLVQSGGGLVQPGGSLKLSCAASGLIFSFNALGWYRQAPGKQRELVAHITSGGSTNYADSVKGRFTISRDNVKKTAFLQMNSLKPEDTAVYYCRSSQSGVEYWGQGTQVTVSS

SEQ ID NO: 67 - protein, 249 aa, synthetic construct
QVQLVQSGGGLVQPGGSLKLSCAASGLIFSFNALGWYRQAPGKQRELVAHITSGGSTNYADSVKGRFTISRDNVKKTAFLQMNSLKPEDTAVYYCRSSQSGVEYWGQGTQVTVSSAAAGGGSGGGSGGGSGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 68 - protein, 256 aa, synthetic construct
QVQLVQSGGGLVQPGGSLKLSCAASGLIFSFNALGWYRQAPGKQRELVAHITSGGSTNYADSVKGRFTISRDNVKKTAFLQMNSLKPEDTAVYYCRSSQSGVEYWGQGTQVTVSSGGGSVSQTSKLTRAETVFPDVDGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 69 - protein, 256 aa, synthetic construct
QVQLVQSGGGLVQPGGSLKLSCAASGLIFSFNALGWYRQAPGKQRELVAHITSGGSTNYADSVKGRFTISRDNVKKTAFLQMNSLKPEDTAVYYCRSSQSGVEYWGQGTQVTVSSGGGSTQSFNDFTRVVGGEDAKPGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 70 - protein, 256 aa, synthetic construct
QVQLVQSGGGLVQPGGSLKLSCAASGLIFSFNALGWYRQAPGKQRELVAHITSGGSTNYADSVKGRFTISRDNVKKTAFLQMNSLKPEDTAVYYCRSSQSGVEYWGQGTQVTVSSGGGSVSQTSKLTRVVGGEDAKPGGGSEVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSPSSQGTLVTVSS

SEQ ID NO: 71 - protein, 609 aa, Homo sapiens [sequence omitted]